(19) United States
(12) Patent Application Publication
(10) Pub. No.: US 2026/0297074 A1
(43) Pub. Date: Oct. 1, 2026

(54) CRYSTALLINE FORMS OF 1-METHYL-1H-INDAZOLE COMPOUNDS AND PREPARATION METHODS AND USES THEREOF

(71) Applicant: Shanghai Fosun Pharmaceutical Industrial Development Co., Ltd., Shanghai (CN)

(72) Inventors: Xinhai CHEN, Shanghai (CN); Pengfei ZHANG, Shanghai (CN); Qingyun JIANG, Shanghai (CN); Fei HAO, Shanghai (CN); Peng LI, Shanghai (CN)

(21) Appl. No.: 19/630,224

(22) Filed: Mar. 26, 2026

(30) Foreign Application Priority Data
Mar. 27, 2025 (CN) 202510375963.8

(51) Int. Cl. C07D 413/12; A61K 31/553

(52) U.S. Cl. CPC C07D 413/12; A61K 31/553

(57) ABSTRACT

The present invention discloses crystalline forms of 1-methyl-1H-indazole compounds, and preparation methods and uses thereof. Specifically, the present invention relates to crystalline forms of the compound of formula (I) and preparation methods thereof.

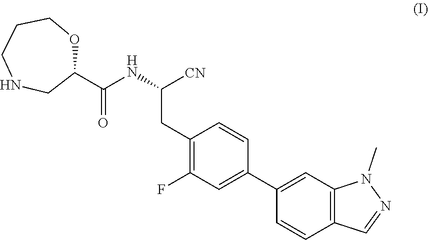 [SMILES: Cn1ncc2ccc(-c3ccc(C[C@@H](C#N)NC(=O)[C@@H]4CNCCCO4)c(F)c3)cc21]

### CROSS-REFERENCE TO RELATED APPLICATIONS

This application claims the benefit of priority to Chinese Application No. 202510375963.8, filed Mar. 27, 2025, the content of which is incorporated herein by reference in its entirety.

### TECHNICAL FIELD

The present invention relates to crystalline forms of 1-methyl-1H-indazole compounds, and preparation methods and uses thereof. Specifically, the present invention relates to crystalline forms of the compound of formula (I) and preparation methods thereof.

### BACKGROUND OF THE INVENTION

Dipeptidyl peptidase 1 (DPP1), also called cathepsin C, is highly expressed in tissues such as lung, kidney, liver, and spleen. DPP1 is a lysosomal cysteine protease that exists as a tetramer composed of four identical subunits, each subunit consisting of a heavy chain, a light chain, and an exclusive domain. The main physiological function of DPP1 is to activate pro-inflammatory neutrophil serine proteases (NSPs, including neutrophil elastase, proteinase 3, and cathepsin G) in the bone marrow by cleaving N-terminal dipeptides. NSPs are closely involved in the regulation of inflammation, can activate various cytokines, and play an important role in the clearance of pathogenic microorganisms. Studies have shown that in patients with diseases such as chronic obstructive pulmonary disease (COPD) or bronchiectasis, persistent inflammatory responses and excessive activation of NSPs are commonly present in the airways, leading to degradation of elastin in the lungs and the like, and further resulting in lung tissue damage and destruction of the bronchial wall tissue. DPP1 inhibitors can inhibit, at the source, the activation of pro-inflammatory neutrophil proteases, thereby suppressing the inflammatory response and airway damage caused by neutrophils in the airway.

While DPP1 inhibitors are often initially evaluated for their activity when dissolved in solution, solid state characteristics such as polymorphism are also important. Polymorphic forms of a drug substance, such as a DPP1 inhibitor, can have different physical properties, including melting point, apparent solubility, dissolution rate, optical and mechanical properties, vapor pressure, and density. These properties can have a direct effect on the ability to process or manufacture a drug substance and the drug product.

### SUMMARY OF THE INVENTION

In one aspect, described herein is a crystalline form of the compound of formula (I), wherein the crystalline form is crystalline form A, crystalline form B, crystalline form C, crystalline form D, or crystalline form E;

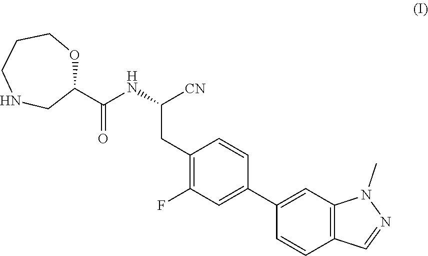 [SMILES: Cn1ncc2ccc(-c3ccc(C[C@@H](C#N)NC(=O)[C@@H]4CNCCCO4)c(F)c3)cc21]

wherein an X-ray powder diffraction pattern of the crystalline form A has diffraction peaks at the following 2θ angles: 13.822±0.200°, 15.236±0.200°, 16.899±0.200°, and 20.673±0.200°, as measured using Cu (Kα) radiation;
an X-ray powder diffraction pattern of the crystalline form B has diffraction peaks at the following 2θ angles: 8.954±0.200°, 13.530±0.200°, 15.480±0.200°, and 18.122±0.200°, as measured using Cu (Kα) radiation;
an X-ray powder diffraction pattern of the crystalline form C has diffraction peaks at the following 2θ angles: 8.069±0.200°, 11.606±0.200°, 13.120±0.200°, and 19.812±0.200°, as measured using Cu (Kα) radiation;
an X-ray powder diffraction pattern of the crystalline form D has diffraction peaks at the following 2θ angles: 9.105±0.200°, 15.206±0.200°, and 16.904±0.200°, as measured using Cu (Kα) radiation;
an X-ray powder diffraction pattern of the crystalline form E has diffraction peaks at the following 2θ angles: 9.331±0.200°, 12.051±0.200°, and 15.214±0.200°, as measured using Cu (Kα) radiation.

In some embodiments, the crystalline form is crystalline form A.

In some embodiments, the X-ray powder diffraction pattern of the crystalline form has diffraction peaks at the following 2θ angles: 11.716±0.200°, 13.822±0.200°, 15.236±0.200°, 16.899±0.200°, 20.673±0.200°, and 24.134±0.200°, as measured using Cu (Kα) radiation.

In some embodiments, the X-ray powder diffraction pattern of the crystalline form has diffraction peaks at the following 2θ angles: 10.267±0.200°, 11.716±0.200°, 13.822±0.200°, 15.236±0.200°, 16.899±0.200°, 17.550±0.200°, 18.110±0.200°, 20.673±0.200°, 24.134±0.200°, as measured using Cu (Kα) radiation.

In some embodiments, the X-ray powder diffraction pattern of the crystalline form has diffraction peaks at the following 2θ angles: 10.267±0.200°, 11.716±0.200°, 13.822±0.200°, 15.236±0.200°, 16.899±0.200°, 17.550±0.200°, 18.110±0.200°, 19.863±0.200°, 20.673±0.200°, 23.025±0.200°, 23.513±0.200°, and 24.134±0.200°, as measured using Cu (Kα) radiation.

In some embodiments, the crystalline form A has one or more characteristics selected from the group consisting of:
an XRPD pattern substantially as shown in FIG. 1;
a DSC pattern substantially as shown in FIG. 2; and/or
a TGA pattern substantially as shown in FIG. 3.

In some embodiments, the crystalline form A has one or more characteristics selected from the group consisting of:
a differential scanning calorimetry curve having a peak value of an endothermic peak at 141.01±3° C.; and/or
a thermogravimetric analysis curve showing a weight loss of 0.040% at 150.0±3° C.

In another aspect, described herein is a crystalline form of the compound of formula (I), wherein the crystalline form is crystalline form A;

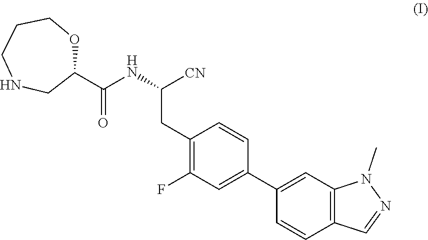 [SMILES: Cn1ncc2ccc(-c3ccc(C[C@@H](C#N)NC(=O)[C@@H]4CNCCCO4)c(F)c3)cc21]

wherein the crystalline form A is characterized as having X-ray powder diffraction pattern with peaks at the following 2θ angles: 13.822±0.200°, 15.236±0.200°, 16.899±0.200°, and 20.673±0.200°, as measured using Cu (Kα) radiation; and one or both of the following:
a differential scanning calorimetry curve having a peak value of an endothermic peak at 141.01±3° C.; or
a thermogravimetric analysis curve showing a weight loss of 0.040% at 150.0±3° C.

In some embodiments, the crystalline form A is characterized as having X-ray powder diffraction pattern with peaks at the following 2θ angles: 13.822±0.200°, 15.236±0.200°, 16.899±0.200°, and 20.673±0.200°, as measured using Cu (Kα) radiation; and a differential scanning calorimetry curve having a peak value of an endothermic peak at 141.01±3° C.

In some embodiments, the crystalline form A is characterized as having X-ray powder diffraction pattern with peaks at the following 2θ angles: 13.822±0.200°, 15.236±0.200°, 16.899±0.200°, and 20.673±0.200°, as measured using Cu (Kα) radiation; and a thermogravimetric analysis curve showing a weight loss of 0.040% at 150.0±3° C.

In another aspect, described herein is a crystalline form of the compound of formula (I), wherein the crystalline form is crystalline form A;

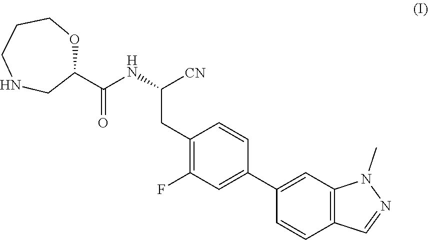 [SMILES: Cn1ncc2ccc(-c3ccc(C[C@@H](C#N)NC(=O)[C@@H]4CNCCCO4)c(F)c3)cc21]

wherein the crystalline form A is characterized as having an XRPD pattern substantially as shown in FIG. 1; and
a DSC pattern substantially as shown in FIG. 2; and/or
a TGA pattern substantially as shown in FIG. 3.

In some embodiments, the crystalline form A is characterized as having an XRPD pattern substantially as shown in FIG. 1; and a DSC pattern substantially as shown in FIG. 2.

In some embodiments, the crystalline form A is characterized as having an XRPD pattern substantially as shown in FIG. 1; and a TGA pattern substantially as shown in FIG. 3.

In a further aspect, described herein is a pharmaceutical composition comprising the crystalline form of the compound of formula (I), and a pharmaceutically acceptable carrier. In some embodiments, the pharmaceutical composition is a solid form pharmaceutical composition. In some embodiments, the solid form pharmaceutical composition is a tablet, pill, or capsule. In some embodiments, the crystalline form of the compound of formula (I) is crystalline form A. In some embodiments, the crystalline form of the compound of formula (I) is crystalline form A that is substantially free of other crystalline forms of the compound of formula (I). In some embodiments, the crystalline form of the compound of formula (I) is crystalline form B. In some embodiments, the crystalline form of the compound of formula (I) is crystalline form C. In some embodiments, the crystalline form of the compound of formula (I) is crystalline form D. In some embodiments, the crystalline form of the compound of formula (I) is crystalline form E.

In one aspect, described herein is a use of the crystalline form of the compound of formula (I) or the pharmaceutical composition comprising the crystalline form of the compound of formula (I) in the preparation of a DPP1 inhibitor or a medicament for preventing or treating a DPP1-related disease.

In one aspect, described herein is a method for preparing the crystalline form of the compound of formula (I) comprising the steps of:
stirring the compound of formula (I) in a mixed solvent of isopropanol and water until a solid precipitates, filtering the solid, and drying the solid at 20-45° C. (preferably 30-40° C.) to obtain the crystalline form A.

Also described herein is the use of the crystalline form of the compound of formula (I) in the treatment of an airway inflammatory disease in a human. Also described herein is the use of a pharmaceutical composition comprising the crystalline form of the compound of formula (I) in the treatment of an airway inflammatory disease in a human. In some embodiments, the crystalline form of the compound of formula (I) is crystalline form A. In some embodiments, the crystalline form of the compound of formula (I) is crystalline form A that is substantially free of other crystalline forms of the compound of formula (I). In some embodiments, the crystalline form of the compound of formula (I) is crystalline form B. In some embodiments, the crystalline form of the compound of formula (I) is crystalline form C. In some embodiments, the crystalline form of the compound of formula (I) is crystalline form D. In some embodiments, the crystalline form of the compound of formula (I) is crystalline form E.

In some embodiments, the airway inflammatory disease is chronic obstructive pulmonary disease (COPD), bronchiectasis, asthma, acute respiratory distress syndrome, exercise-induced bronchoconstriction (EIB), chronic bronchitis, emphysema, alpha-1 antitrypsin deficiency, combined pulmonary fibrosis and emphysema (CPFE), or asthma-COPD overlap syndrome (ACOS).

In some embodiments, the asthma is allergic asthma, aspirin-induced asthma, cough-variant asthma, exercise-induced asthma, nighttime asthma, steroid-resistant asthma, occupational asthma, eosinophilic asthma, neutrophilic asthma, or mixed eosinophilic and neutrophilic asthma.

In some embodiments, the airway inflammatory disease is chronic obstructive pulmonary disease (COPD).

In some embodiments, the airway inflammatory disease is bronchiectasis.

Also described herein is a method of treating an airway inflammatory disease in a human comprising administering to the human in need thereof a therapeutically effective amount of the crystalline form of the compound of formula (I). In some embodiments, the crystalline form of the compound of formula (I) is crystalline form A. In some embodiments, the crystalline form of the compound of formula (I) is crystalline form A that is substantially free of other crystalline forms of the compound of formula (I). In some embodiments, the crystalline form of the compound of formula (I) is crystalline form B. In some embodiments, the crystalline form of the compound of formula (I) is crystalline form C. In some embodiments, the crystalline form of the compound of formula (I) is crystalline form D. In some embodiments, the crystalline form of the compound of formula (I) is crystalline form E. In some embodiments, the therapeutically effective amount of the crystalline form is administered in the form of a solid form pharmaceutical composition. In some embodiments, the solid form pharmaceutical composition is a tablet, pill, or capsule.

In some embodiments, the airway inflammatory disease is chronic obstructive pulmonary disease (COPD), bronchiectasis, asthma, acute respiratory distress syndrome, exercise-induced bronchoconstriction (EIB), chronic bronchitis, emphysema, alpha-1 antitrypsin deficiency, combined pulmonary fibrosis and emphysema (CPFE), or asthma-COPD overlap syndrome (ACOS).

In some embodiments, the asthma is allergic asthma, aspirin-induced asthma, cough-variant asthma, exercise-induced asthma, nighttime asthma, steroid-resistant asthma, occupational asthma, eosinophilic asthma, neutrophilic asthma, or mixed eosinophilic and neutrophilic asthma.

In some embodiments, the airway inflammatory disease is chronic obstructive pulmonary disease (COPD).

In some embodiments, the airway inflammatory disease is bronchiectasis.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1: XRPD pattern of the crystalline form A of the compound of formula (I).

FIG. 2: DSC pattern of the crystalline form A of the compound of formula (I).

FIG. 3: TGA pattern of the crystalline form A of the compound of formula (I).

FIG. 4: XRPD pattern of the crystalline form B of the compound of formula (I).

FIG. 5: DSC pattern of the crystalline form B of the compound of formula (I).

FIG. 6: TGA pattern of the crystalline form B of the compound of formula (I).

FIG. 7: XRPD pattern of the crystalline form C of the compound of formula (I).

FIG. 8: DSC pattern of the crystalline form C of the compound of formula (I).

FIG. 9: TGA pattern of the crystalline form C of the compound of formula (I).

FIG. 10: XRPD pattern of the crystalline form D of the compound of formula (I).

FIG. 11: DSC pattern of the crystalline form D of the compound of formula (I).

FIG. 12: TGA pattern of the crystalline form D of the compound of formula (I).

FIG. 13: XRPD pattern of the crystalline form E of the compound of formula (I).

FIG. 14: DSC pattern of the crystalline form E of the compound of formula (I).

FIG. 15: TGA pattern of the crystalline form E of the compound of formula (I).

FIG. 16: DVS pattern of the crystalline form A of the compound of formula (I).

### DETAILED DESCRIPTION OF THE INVENTION

In airway inflammatory disorders, activated neutrophils are important disease drivers as they release tissue-damaging neutrophil serine proteases (NSPs) that promote airway inflammation, mucus plugging, and exacerbations. Dipeptidyl peptidase 1 (DPP1, also called cathepsin C) enzymatically activates NSPs, including neutrophil elastase (NE), proteinase 3 (PR3), and cathepsin G (CatG) during neutrophil maturation in the bone marrow; hence DPP1 inhibitors may reduce systemic NSP levels. NSPs in the airway reduce cilia oscillation frequency, hinder the clearance of apoptotic cells, and promote sputum production. NSPs also degrade extracellular matrix components contributing to alveolar destruction and chronic airway remodeling and degrade antimicrobial peptides causing increased bacterial colonization and lung infection.

DPP1 is required in neutrophils for the activation of NSPs such as NE, CatG, and PR3 which are key drivers of disease in airway inflammatory disorders such as chronic obstructive pulmonary disease (COPD) and bronchiectasis. Excess NSPs promote airway inflammation, mucus plugging, and exacerbations (ie, symptom flare ups). Neutrophils and NSPs are further increased during exacerbations and promote a feedforward cycle of pathology. Hence, drugs that block or reduce NSPs have therapeutic potential for controlling lung inflammatory diseases, with critical unmet need in COPD.

COPD is a chronic inflammatory disease that impacts the airways and alveoli. COPD causes significant morbidity and is the third leading cause of death worldwide (see 2025 Global Initiative for Chronic Obstructive Lung Disease [GOLD] Report). Patients with COPD develop chronic bronchitis and emphysema and are vulnerable to frequent bacterial infections. The pathophysiology of COPD includes airflow limitation and abnormal gas exchange which may be accompanied by mucus hypersecretion and airway epithelial ciliary dysfunction. Tobacco smoking and environmental inhalational exposure to particulate matter and noxious gases are the most common causes of COPD.

While available treatments for COPD (e.g., inhaled short-acting bronchodilators (short acting beta agonists, SABA and short acting muscarinic agonists, SAMA), long-acting bronchodilators/muscarinic agonists (LABA/LAMA), inhaled corticosteroids (ICS), phosphodiesterase 3 (PDE) 3 and PDE4 inhibitors, dupilumab, mepolizumab) can reduce the rate of exacerbations and improve quality of life, they have little impact on COPD progression and mortality. Therefore, there is a significant need for new drugs with novel mechanisms of action or new routes of administration to address the unmet medical demands for controlling COPD. DPP1 inhibition by the compound of formula (I) represents a promising therapeutic strategy for reducing NSP activity levels and improving outcomes in COPD, an effect that is supported by the approval of the DPP1 inhibitor brensocatib for the treatment of bronchiectasis, an airway inflammatory disease with similarities to COPD.

Non-cystic fibrosis bronchiectasis (NCFBE), also known as bronchiectasis, is caused by recurrent suppurative infections of various etiologies, which lead to repeated damage and/or obstruction of small and medium-sized bronchi and destruction of the bronchial structure, thus causing persistent, abnormal bronchial dilatation. The etiologies are complex and diverse, including persistent bacterial infection, dysregulated immune response, impaired mucociliary clearance system, abnormal bronchial dilatation caused by airway mucus obstruction, and chronic inflammation. Clinical manifestations include persistent or recurrent coughing and sputum, sometimes accompanied by hemoptysis, which can lead to respiratory dysfunction and chronic core pulmonale.

Bronchiectasis is a common chronic respiratory tract disorder with a long course and irreversible lesions. It can seriously damage the patient's lung tissue and function, affect the patient's quality of life, and cause a heavy socioeconomic burden. Bronchiectasis combined with other lung disorders is also a concern. Approximately 15% to 30% of patients with chronic bronchitis or COPD have bronchiectasis lesions, and up to 50% of patients with bronchiectasis have severe COPD.

The current clinical standard of care for bronchiectasis includes non-drug therapy, drug therapy, and surgical therapy. Despite the recommended treatments outlined in the guidelines, patients with bronchiectasis still experience repeated acute exacerbations. There is an urgent need for novel therapeutic strategies aimed at slowing disease progression, reducing the frequency of acute exacerbations, and enhancing lung function.

After extensive and in-depth research, and through substantial screening and testing, the inventors have, for the first time, provided novel crystalline forms of the compound of formula (I), as well as preparation methods and uses thereof. On this basis, the present invention has been accomplished.

### The Compound of Formula (I)

The compound of formula (I) is an orally active, small molecule inhibitor of the enzyme dipeptidyl peptidase 1 (DPP1). As compared with other drug classes for the treatment of airway inflammation, the compound of formula (I) is anticipated to avoid the resistance issues associated with antibiotics and the safety risks associated with corticosteroids. In some embodiments, administration of the compound of formula (I) reduces the frequency of acute exacerbations seen in COPD and bronchiectasis, slow the progression of airway dilation, improve clinical symptoms and quality of life for patients, and is expected to fill the gap in long-term treatment drugs for these indications.

The present invention aims to provide novel crystalline forms of the compound of formula (I), wherein the crystalline form is crystalline form A, crystalline form B, crystalline form C, crystalline form D, or crystalline form E.

The present invention provides the crystalline form A of the compound of formula (I), and its X-ray powder diffraction pattern has characteristic diffraction peaks at the following 2θ angles: 13.822±0.200°, 15.236±0.200°, 16.899±0.200°, and 20.673±0.200°;

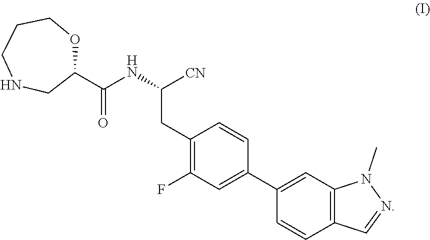 [SMILES: Cn1ncc2ccc(-c3ccc(C[C@@H](C#N)NC(=O)[C@@H]4CNCCCO4)c(F)c3)cc21]

In some embodiments of the present invention, the X-ray powder diffraction pattern of the above crystalline form A has characteristic diffraction peaks at the following 2θ angles: 13.822±0.200°, 15.236±0.200°, 16.899±0.200°, 20.673±0.200°, and 24.134±0.200°.

In some embodiments of the present invention, the X-ray powder diffraction pattern of the above crystalline form A has characteristic diffraction peaks at the following 2θ angles: 11.716±0.200°, 13.822±0.200°, 15.236±0.200°, 16.899±0.200°, 20.673±0.200°, and 24.134±0.200°.

In some embodiments of the present invention, the X-ray powder diffraction pattern of the above crystalline form A has characteristic diffraction peaks at the following 2θ angles: 10.267±0.200°, 11.716±0.200°, 13.822±0.200°, 15.236±0.200°, 16.899±0.200°, 17.550±0.200°, 18.110±0.200°, 20.673±0.200°, 24.134±0.200°.

In some embodiments of the present invention, the X-ray powder diffraction pattern of the above crystalline form A has characteristic diffraction peaks at the following 2θ angles: 10.267±0.200°, 11.716±0.200°, 13.822±0.200°, 15.236±0.200°, 16.899±0.200°, 17.550±0.200°, 18.110±0.200°, 20.673±0.200°, 23.513±0.200°, 24.134±0.200°.

In some embodiments of the present invention, the X-ray powder diffraction pattern of the above crystalline form A has characteristic diffraction peaks at the following 2θ angles: 10.267±0.200°, 11.716±0.200°, 13.822±0.200°, 15.236±0.200°, 16.899±0.200°, 17.550±0.200°, 18.110±0.200°, 19.863±0.200°, 20.673±0.200°, 23.025±0.200°, 23.513±0.200°, and 24.134±0.200°.

In some embodiments of the present invention, the X-ray powder diffraction pattern of the above crystalline form A has characteristic diffraction peaks at the following 2θ angles: 7.672±0.200°, 10.267±0.200°, 11.716±0.200°, 13.822±0.200°, 15.236±0.200°, 16.899±0.200°, 17.550±0.200°, 18.110±0.200°, 18.484±0.200°, 19.863±0.200°, 20.673±0.200°, 23.025±0.200°, 23.513±0.200°, 24.134±0.200°, 25.621±0.200°, and 26.924±0.200°.

In some embodiments of the present invention, the X-ray powder diffraction pattern of the above crystalline form A has characteristic diffraction peaks at the following 2θ angles: 7.672±0.100°, 10.267±0.100°, 11.716±0.100°, 13.822±0.100°, 15.236±0.100°, 16.899±0.100°, 17.550±0.100°, 18.110±0.100°, 18.484±0.100°, 19.863±0.100°, 20.673±0.100°, 23.025±0.100°, 23.513±0.100°, 24.134±0.100°, 25.621±0.100°, and 26.924±0.100°.

In some embodiments of the present invention, the X-ray powder diffraction pattern of the above crystalline form A has characteristic diffraction peaks at the following 2θ angles: 5.889±0.200°, 6.954±0.200°, 7.672±0.200°, 9.110±0.200°, 10.267±0.200°, 11.716±0.200°, 13.367±0.200°, 13.822±0.200°, 15.236±0.200°, 16.899±0.200°, 17.550±0.200°, 18.110±0.200°, 18.484±0.200°, 18.813±0.200°, 19.863±0.200°, 20.673±0.200°, 21.748±0.200°, 22.433±0.200°, 23.025±0.200°, 23.513±0.200°, 24.134±0.200°, 24.563±0.200°, 25.621±0.200°, 26.403±0.200°, 26.924±0.200°, 27.775±0.200°, 28.336±0.200°, 29.197±0.200°, 30.076±0.200°, 30.822±0.200°, 31.926±0.200°, 33.208±0.200°, 33.423±0.200°, 34.209±0.200°, 34.748±0.200°, 35.427±0.200°, 36.157±0.200°, 36.701±0.200°, 37.391±0.200°, 38.174±0.200°, 38.426±0.200°, 39.734±0.200°, 39.988±0.200°, 40.731±0.200°, 41.079±0.200°, 42.194±0.200°, 43.590±0.200°, 44.030±0.200°, 44.489±0.200°, 45.700±0.200°, 46.652±0.200°, 46.968±0.200°, 48.017±0.200°, 49.638±0.200°, 50.604±0.200°, 54.563±0.200°, 55.180±0.200°, 56.460±0.200°, 57.287±0.200°.

In some embodiments of the present invention, the X-ray powder diffraction pattern of the above crystalline form A has characteristic diffraction peaks at the following 2θ angles: 5.889±0.100°, 6.954±0.100°, 7.672±0.100°, 9.110±0.100°, 10.267±0.100°, 11.716±0.100°, 13.367±0.100°, 13.822±0.100°, 15.236±0.100°, 16.899±0.100°, 17.550±0.100°, 18.110±0.100°, 18.484±0.100°, 18.813±0.100°, 19.863±0.100°, 20.673±0.100°, 21.748±0.100°, 22.433±0.100°, 23.025±0.100°, 23.513±0.100°, 24.134±0.100°, 24.563±0.100°, 25.621±0.100°, 26.403±0.100°, 26.924±0.100°, 27.775±0.100°, 28.336±0.100°, 29.197±0.100°, 30.076±0.100°, 30.822±0.100°, 31.926±0.100°, 33.208±0.100°, 33.423±0.100°, 34.209±0.100°, 34.748±0.100°, 35.427±0.100°, 36.157±0.100°, 36.701±0.100°, 37.391±0.100°, 38.174±0.100°, 38.426±0.100°, 39.734±0.100°, 39.988±0.100°, 40.731±0.100°, 41.079±0.100°, 42.194±0.100°, 43.590±0.100°, 44.030±0.100°, 44.489±0.100°, 45.700±0.100°, 46.652±0.100°, 46.968±0.100°, 48.017±0.100°, 49.638±0.100°, 50.604±0.100°, 54.563±0.100°, 55.180±0.100°, 56.460±0.100°, 57.287±0.100°.

In some embodiments of the present invention, the X-ray powder diffraction pattern of the above crystalline form A has characteristic diffraction peaks at the following 2θ angles: 13.822±0.200°, 15.236±0.200°, 16.899±0.200°, and/or 20.673±0.200°, and/or 5.889±0.200°, and/or 6.954±0.200°, and/or 7.672±0.200°, and/or 9.110±0.200°, and/or 10.267±0.200°, and/or 11.716±0.200°, and/or 13.367±0.200°, and/or 17.550±0.200°, and/or 18.110±0.200°, and/or 18.484±0.200°, and/or 18.813±0.200°, and/or 19.863±0.200°, and/or 21.748±0.200°, and/or 22.433±0.200°, and/or 23.025±0.200°, and/or 23.513±0.200°, and/or 24.134±0.200°, and/or 24.563±0.200°, and/or 25.621±0.200°, and/or 26.403±0.200°, and/or 26.924±0.200°, and/or 27.775±0.200°, and/or 28.336±0.200°, and/or 29.197±0.200°, and/or 30.076±0.200°, and/or 30.822±0.200°, and/or 31.926±0.200°, and/or 33.208±0.200°, and/or 33.423±0.200°, and/or 34.209±0.200°, and/or 34.748±0.200°, and/or 35.427±0.200°, and/or 36.157±0.200°, and/or 36.701±0.200°, and/or 37.391±0.200°, and/or 38.174±0.200°, and/or 38.426±0.200°, and/or 39.734±0.200°, and/or 39.988±0.200°, and/or 40.731±0.200°, and/or 41.079±0.200°, and/or 42.194±0.200°, and/or 43.590±0.200°, and/or 44.030±0.200°, and/or 44.489±0.200°, and/or 45.700±0.200°, and/or 46.652±0.200°, and/or 46.968±0.200°, and/or 48.017±0.200°, and/or 49.638±0.200°, and/or 50.604±0.200°, and/or 54.563±0.200°, and/or 55.180±0.200°, and/or 56.460±0.200°, and/or 57.287±0.200°.

In some embodiments of the present invention, the X-ray powder diffraction pattern of the above crystalline form A has characteristic diffraction peaks at the following 2θ angles: 13.822±0.100°, 15.236±0.100°, 16.899±0.100°, and/or 20.673±0.100°, and/or 5.889±0.100°, and/or 6.954±0.100°, and/or 7.672±0.100°, and/or 9.110±0.100°, and/or 10.267±0.100°, and/or 11.716±0.100°, and/or 13.367±0.100°, and/or 17.550±0.100°, and/or 18.110±0.100°, and/or 18.484±0.100°, and/or 18.813±0.100°, and/or 19.863±0.100°, and/or 21.748±0.100°, and/or 22.433±0.100°, and/or 23.025±0.100°, and/or 23.513±0.100°, and/or 24.134±0.100°, and/or 24.563±0.100°, and/or 25.621±0.100°, and/or 26.403±0.100°, and/or 26.924±0.100°, and/or 27.775±0.100°, and/or 28.336±0.100°, and/or 29.197±0.100°, and/or 30.076±0.100°, and/or 30.822±0.100°, and/or 31.926±0.100°, and/or 33.208±0.100°, and/or 33.423±0.100°, and/or 34.209±0.100°, and/or 34.748±0.100°, and/or 35.427±0.100°, and/or 36.157±0.100°, and/or 36.701±0.100°, and/or 37.391±0.100°, and/or 38.174±0.100°, and/or 38.426±0.100°, and/or 39.734±0.100°, and/or 39.988±0.100°, and/or 40.731±0.100°, and/or 41.079±0.100°, and/or 42.194±0.100°, and/or 43.590±0.100°, and/or 44.030±0.100°, and/or 44.489±0.100°, and/or 45.700±0.100°, and/or 46.652±0.100°, and/or 46.968±0.100°, and/or 48.017±0.100°, and/or 49.638±0.100°, and/or 50.604±0.100°, and/or 54.563±0.100°, and/or 55.180±0.100°, and/or 56.460±0.100°, and/or 57.287±0.100°.

In some embodiments of the present invention, the X-ray powder diffraction pattern of the above crystalline form A has characteristic diffraction peaks at the following 2θ angles: 5.889°, 6.954°, 7.672°, 9.110°, 10.267°, 11.716°, 13.367°, 13.822°, 15.236°, 16.899°, 17.550°, 18.110°, 18.484°, 18.813°, 19.863°, 20.673°, 21.748°, 22.433°, 23.025°, 23.513°, 24.134°, 24.563°, 25.621°, 26.403°, 26.924°, 27.775°, 28.336°, 29.197°, 30.076°, 30.822°, 31.926°, 33.208°, 33.423°, 34.209°, 34.748°, 35.427°, 36.157°, 36.701°, 37.391°, 38.174°, 38.426°, 39.734° 39.988°, 40.731°, 41.079°, 42.194°, 43.590°, 44.030°, 44.489°, 45.700°, 46.652°, 46.968°, 48.017°, 49.638°, 50.604°, 54.563°, 55.180°, 56.460°, and 57.287°.

In some embodiments of the present invention, the XRPD pattern analysis data of the above crystalline form A is as shown in Table 1.

TABLE 1
XRPD analysis data of the crystalline form A of the compound of formula (I)

| | 2θ | Peak | Interplanar | Relative | | | Peak | | Relative |
|---|---|---|---|---|---|---|---|---|---|
| | angle | height | spacing | intensity | | 2θ angle | height | d Value | intensity |
| No. | [°] | [cts] | (Å) | (%) | No. | [°] | [cts] | (Å) | (%) |
| 1 | 5.889 | 485.859 | 14.99649 | 0.10% | 31 | 31.926 | 2059.45 | 2.80088 | 2.00% |
| 2 | 6.954 | 686.57 | 12.70179 | 0.30% | 32 | 33.208 | 2591.88 | 2.69569 | 2.80% |
| 3 | 7.672 | 2549.23 | 11.51474 | 3.20% | 33 | 33.423 | 3022.74 | 2.67880 | 3.50% |
| 4 | 9.110 | 848.602 | 9.69979 | 0.30% | 34 | 34.209 | 1976.45 | 2.61901 | 1.80% |
| 5 | 10.267 | 6704.59 | 8.60863 | 9.60% | 35 | 34.748 | 1450.85 | 2.57967 | 0.90% |
| 6 | 11.716 | 9891.48 | 7.54697 | 14.60% | 36 | 35.427 | 2193.48 | 2.53173 | 2.20% |
| 7 | 13.367 | 1377.73 | 6.61833 | 0.90% | 37 | 36.157 | 1214.34 | 2.48228 | 0.70% |
| 8 | 13.822 | 14767.5 | 6.40165 | 22.40% | 38 | 36.701 | 1033.32 | 2.44672 | 0.40% |
| 9 | 15.236 | 14887.4 | 5.81044 | 22.40% | 39 | 37.391 | 1061.05 | 2.40315 | 0.40% |
| 10 | 16.899 | 63124.8 | 5.24233 | 100.00% | 40 | 38.174 | 2151.70 | 2.35564 | 2.10% |
| 11 | 17.550 | 9047.44 | 5.04937 | 12.60% | 41 | 38.426 | 1916.48 | 2.34078 | 1.70% |
| 12 | 18.110 | 12382 | 4.89450 | 18.00% | 42 | 39.734 | 2626.01 | 2.26667 | 2.80% |
| 13 | 18.484 | 6709.56 | 4.79616 | 8.80% | 43 | 39.988 | 2283.73 | 2.25287 | 2.30% |
| 14 | 18.813 | 3221.39 | 4.71304 | 3.20% | 44 | 40.731 | 2478.90 | 2.21347 | 2.70% |
| 15 | 19.863 | 5976.79 | 4.46636 | 7.60% | 45 | 41.079 | 1139.71 | 2.19553 | 0.60% |
| 16 | 20.673 | 32059.7 | 4.29299 | 49.60% | 46 | 42.194 | 1228.22 | 2.14004 | 0.80% |
| 17 | 21.748 | 4119.64 | 4.08319 | 4.50% | 47 | 43.590 | 894.091 | 2.07469 | 0.30% |
| 18 | 22.433 | 2562.04 | 3.96011 | 1.90% | 48 | 44.030 | 1391.09 | 2.05497 | 1.10% |
| 19 | 23.025 | 7734.99 | 3.85962 | 10.30% | 49 | 44.489 | 1482.93 | 2.03482 | 1.20% |
| 20 | 23.513 | 11731 | 3.78059 | 16.70% | 50 | 45.700 | 1246.88 | 1.98366 | 0.80% |
| 21 | 24.134 | 22544.5 | 3.68464 | 34.30% | 51 | 46.652 | 962.897 | 1.94541 | 0.40% |
| 22 | 24.563 | 4352.29 | 3.62131 | 4.90% | 52 | 46.968 | 1304.50 | 1.93301 | 0.90% |
| 23 | 25.621 | 5302.70 | 3.47413 | 6.60% | 53 | 48.017 | 1216.14 | 1.89324 | 0.90% |
| 24 | 26.403 | 4707.89 | 3.37289 | 5.70% | 54 | 49.638 | 832.609 | 1.83513 | 0.30% |
| 25 | 26.924 | 7951.31 | 3.30883 | 11.00% | 55 | 50.604 | 1023.41 | 1.80235 | 0.60% |
| 26 | 27.775 | 1663.64 | 3.20942 | 0.90% | 56 | 54.563 | 833.802 | 1.68056 | 0.40% |
| 27 | 28.336 | 4138.68 | 3.14712 | 5.00% | 57 | 55.180 | 813.397 | 1.66321 | 0.40% |
| 28 | 29.197 | 3540.3 | 3.05618 | 4.10% | 58 | 56.460 | 598.187 | 1.62851 | 0.10% |
| 29 | 30.076 | 2705.51 | 2.96885 | 2.80% | 59 | 57.287 | 631.574 | 1.60693 | 0.20% |
| 30 | 30.822 | 1973.87 | 2.89867 | 1.70% | | | | | |

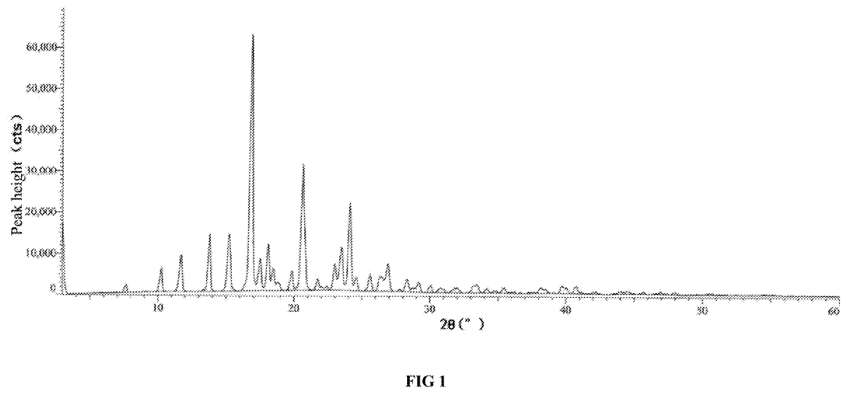

In some embodiments of the present invention, the XR-PD pattern of the above crystalline form A is substantially as shown in FIG. 1.

In some embodiments of the present invention, the differential scanning calorimetry (DSC) curve of the above crystalline form A has a peak value of an endothermic peak at 141.01±3° C.

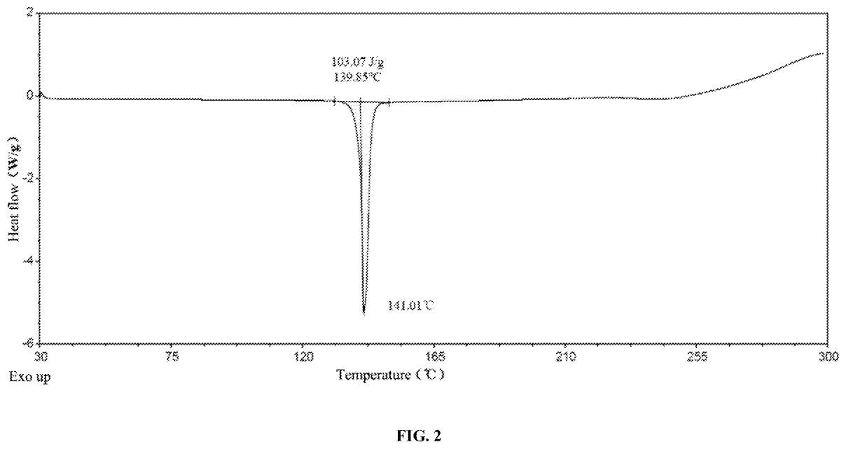

In some embodiments of the present invention, the DSC pattern of the above crystalline form A is substantially as shown in FIG. 2.

In some embodiments of the present invention, the thermogravimetric analysis (TGA) curve of the above crystalline form A shows a weight loss of 0.040% at 150.0±3° C.

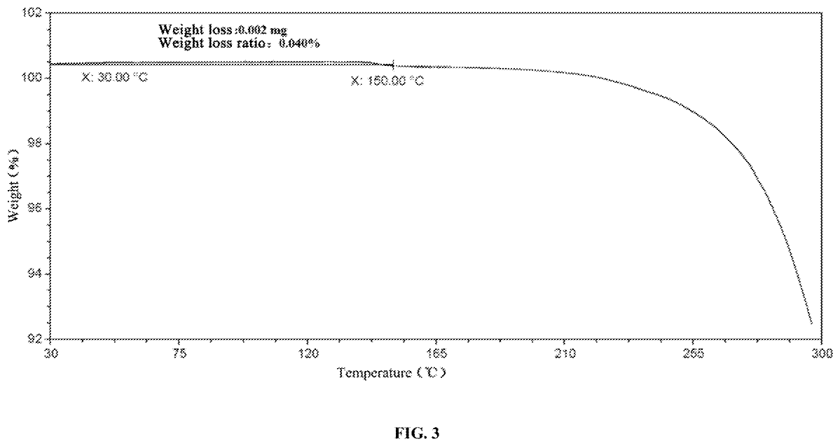

In some embodiments of the present invention, the TGA pattern of the above crystalline form A is substantially as shown in FIG. 3.

In some embodiments of the present invention, the above crystalline form A of the compound of formula (I) is substantially free of other solid state forms of the compound of formula (I). In some embodiments, the above crystalline form A of the compound of formula (I) is substantially free of crystalline form B, crystalline form C, crystalline form D, and/or crystalline form E of the compound of formula (I). In some embodiments, the above crystalline form A of the compound of formula (I) is substantially free of crystalline form B of the compound of formula (I). In some embodiments, the above crystalline form A of the compound of formula (I) is substantially free of crystalline form C of the compound of formula (I). In some embodiments, the above crystalline form A of the compound of formula (I) is substantially free of crystalline form D of the compound of formula (I). In some embodiments, the above crystalline form A of the compound of formula (I) is substantially free of crystalline form E of the compound of formula (I). In some embodiments, the above crystalline form A of the compound of formula (I) comprises less than 0.05%, 0.10%, 0.15%, 0.20%, 0.25%, 0.30%, 0.35%, 0.40%, 0.45%, 0.50%, 0.60%, 0.70%, 0.80%, 0.90%, 1.0%, 1.5%, 2%, 2.5%, 3.0%, 3.5%, 4.0%, 4.5%, or 5.0% (w/w) the crystalline form B, crystalline form C, crystalline form D, and/or crystalline form E of the compound of formula (I). In some embodiments, the above crystalline form A of the compound of formula (I) comprises less than 0.05%, 0.10%, 0.15%, 0.20%, 0.25%, 0.30%, 0.35%, 0.40%, 0.45%, 0.50%, 0.60%, 0.70%, 0.80%, 0.90%, or 1.0% (w/w) the crystalline form B, crystalline form C, crystalline form D, and/or crystalline form E of the compound of formula (I). In some embodiments, the above crystalline form A of the compound of formula (I) comprises not more than 0.05%, 0.10%, 0.15%, 0.20%, 0.25%, 0.30%, 0.35%, 0.40%, 0.45%, 0.50%, 0.60%, 0.70%, 0.80%, 0.90%, 1.0%, 1.5%, 2%, 2.5%, 3.0%, 3.5%, 4.0%, 4.5%, or 5.0% (w/w) the crystalline form B, crystalline form C, crystalline form D, and/or crystalline form E of the compound of formula (I). In some embodiments, the above crystalline form A of the compound of formula (I) comprises not more than 0.05%, 0.10%, 0.15%, 0.20%, 0.25%, 0.30%, 0.35%, 0.40%, 0.45%, 0.50%, 0.60%, 0.70%, 0.80%, 0.90%, or 1.0% (w/w) the crystalline form B, crystalline form C, crystalline form D, and/or crystalline form E of the compound of formula (I). In some embodiments, detection is by XRPD, DSC, TGA, or the like.

In some embodiments of the present invention, the above crystalline form A of the compound of formula (I) is essentially free of other solid state forms of the compound of formula (I). In some embodiments, the above crystalline form A of the compound of formula (I) is essentially free of crystalline form B, crystalline form C, crystalline form D, and/or crystalline form E of the compound of formula (I). In some embodiments, the above crystalline form A of the compound of formula (I) is essentially free of crystalline form B of the compound of formula (I). In some embodiments, the above crystalline form A of the compound of formula (I) is essentially free of crystalline form C of the compound of formula (I). In some embodiments, the above crystalline form A of the compound of formula (I) is essentially free of crystalline form D of the compound of formula (I). In some embodiments, the above crystalline form A of the compound of formula (I) is essentially free of crystalline form E of the compound of formula (I). In some embodiments, the above crystalline form A of the compound of formula (I) comprises no detectable amount of the crystalline form B, crystalline form C, crystalline form D, and/or crystalline form E of the compound of formula (I). In some embodiments, detection is by XRPD, DSC, TGA, or the like.

In some embodiments of the present invention, the above crystalline form A of the compound of formula (I) is free of other solid state forms of the compound of formula (I). In some embodiments, the above crystalline form A of the compound of formula (I) is free of crystalline form B, crystalline form C, crystalline form D, and/or crystalline form E of the compound of formula (I). In some embodiments, the above crystalline form A of the compound of formula (I) is free of crystalline form B of the compound of formula (I). In some embodiments, the above crystalline form A of the compound of formula (I) is free of crystalline form C of the compound of formula (I). In some embodiments, the above crystalline form A of the compound of formula (I) is free of crystalline form D of the compound of formula (I). In some embodiments, the above crystalline form A of the compound of formula (I) is free of crystalline form E of the compound of formula (I). In some embodiments, the above crystalline form A of the compound of formula (I) comprises no detectable amount of the crystalline form B, crystalline form C, crystalline form D, and/or crystalline form E of the compound of formula (I). In some embodiments, detection is by XRPD, DSC, TGA, or the like.

In some embodiments of the present invention, the above crystalline form A is an anhydrous crystalline form.

The present invention further provides the crystalline form B of the compound of formula (I), and its X-ray powder diffraction pattern has characteristic diffraction peaks at the following 2θ angles: 8.954±0.200°, 13.530±0.200°, 15.480±0.200°, and 18.122±0.200°;

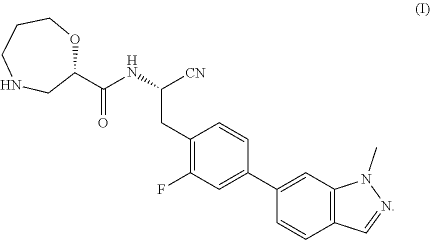 [SMILES: Cn1ncc2ccc(-c3ccc(C[C@@H](C#N)NC(=O)[C@@H]4CNCCCO4)c(F)c3)cc21]

In some embodiments of the present invention, the X-ray powder diffraction pattern of the above crystalline form B has characteristic diffraction peaks at the following 2θ angles: 4.396±0.200°, 7.598±0.200°, 8.954±0.200°, 9.987±0.200°, 13.530±0.200°, 15.054±0.200°, 15.480±0.200°, 17.549±0.200°, 18.122±0.200°, 19.604±0.200°, 22.535±0.200°, 27.395±0.200°, 31.079±0.200°, 35.470±0.200°, and 36.859±0.200°.

In some embodiments of the present invention, the X-ray powder diffraction pattern of the above crystalline form B has characteristic diffraction peaks at the following 2θ angles: 8.954±0.200°, 13.530±0.200°, and/or 15.480±0.200°, and/or 18.122±0.200°, and/or 4.396±0.200°, and/or 7.598±0.200°, and/or 9.987±0.200°, and/or 15.054±0.200°, and/or 17.549±0.200°, and/or 19.604±0.200°, and/or 22.535±0.200°, and/or 27.395±0.200°, and/or 31.079±0.200°, and/or 35.470±0.200°, and/or 36.859±0.200°.

In some embodiments of the present invention, the X-ray powder diffraction pattern of the above crystalline form B has characteristic diffraction peaks at the following 2θ angles: 4.396°, 7.598°, 8.954°, 9.987°, 13.530°, 15.054°, 15.480°, 17.549°, 18.122°, 19.604°, 22.535°, 27.395°, 31.079°, 35.470°, and 36.859°.

In some embodiments of the present invention, the XRPD pattern analysis data of the above crystalline form B is as shown in Table 2.

TABLE 2
XRPD analysis data of the crystalline
form B of the compound of formula (I)

| | 2θ | Peak | Interplanar | Relative |
|---|---|---|---|---|
| | angle | height | spacing | intensity |
| No. | [°2θ] | [cts] | (Å) | (%) |
| 1 | 4.396 | 179.431 | 20.08505 | 11.7 |
| 2 | 7.598 | 69.6230 | 11.62536 | 3.8 |
| 3 | 8.954 | 806.947 | 9.86780 | 100.0 |
| 4 | 9.987 | 86.5460 | 8.84993 | 5.8 |
| 5 | 13.530 | 249.527 | 6.53919 | 28.0 |
| 6 | 15.054 | 77.2947 | 5.88046 | 5.5 |
| 7 | 15.480 | 228.502 | 5.71972 | 25.4 |
| 8 | 17.549 | 70.5828 | 5.04959 | 5.0 |
| 9 | 18.122 | 167.240 | 4.89131 | 17.5 |
| 10 | 19.604 | 74.1768 | 4.52472 | 5.6 |
| 11 | 22.535 | 55.8582 | 3.94233 | 3.5 |
| 12 | 27.395 | 45.1250 | 3.25300 | 2.6 |
| 13 | 31.079 | 32.3125 | 2.87532 | 1.6 |
| 14 | 35.470 | 32.8841 | 2.52874 | 1.8 |
| 15 | 36.859 | 43.7895 | 2.43660 | 3.3 |

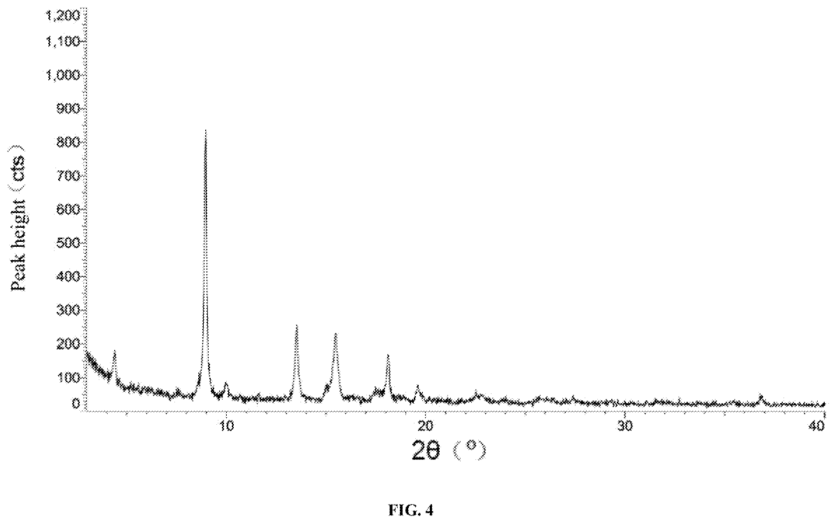

In some embodiments of the present invention, the XRPD pattern of the above crystalline form B is substantially as shown in FIG. 4.

In some embodiments of the present invention, the differential scanning calorimetry curve of the above crystalline form B has a peak value of an endothermic peak at 221.82±3° C.

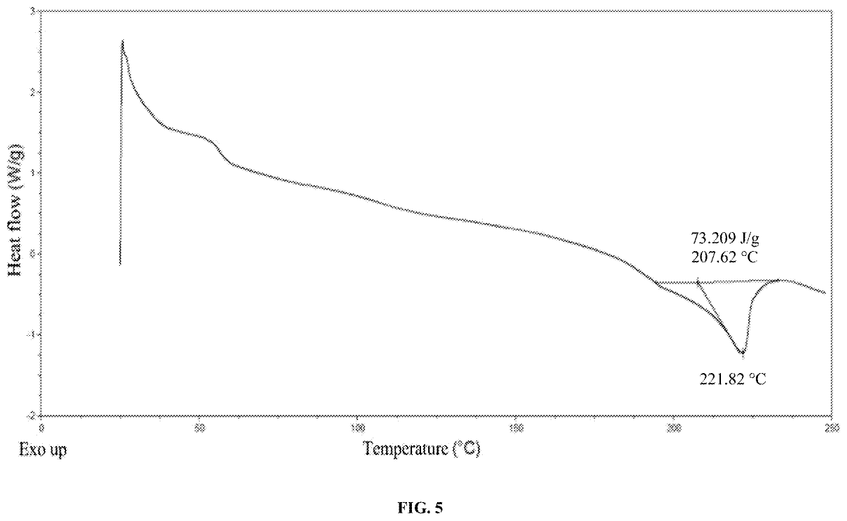

In some embodiments of the present invention, the DSC pattern of the above crystalline form B is substantially as shown in FIG. 5.

In some embodiments of the present invention, the thermogravimetric analysis curve of the above crystalline form B shows a weight loss of 1.056% at 100.00±3° C.

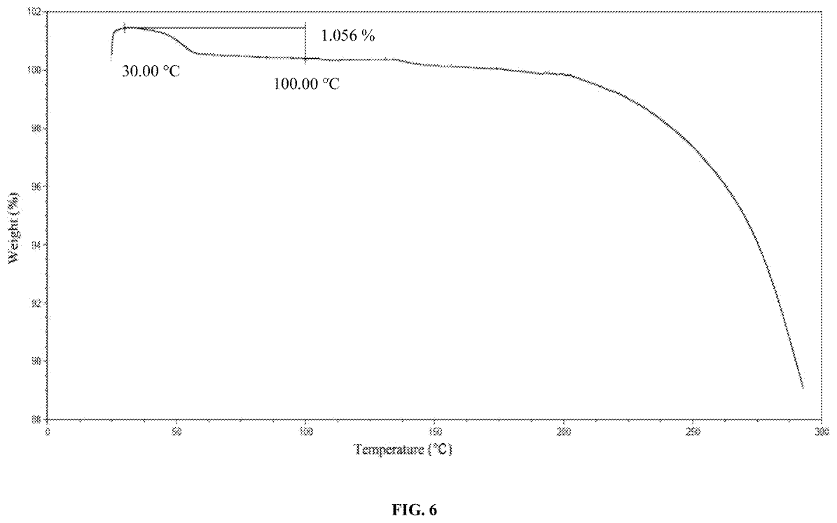

In some embodiments of the present invention, the TGA pattern of the above crystalline form B is substantially as shown in FIG. 6.

The present invention further provides the crystalline form C of the compound of formula (I), and its X-ray powder diffraction pattern has characteristic diffraction peaks at the following 2θ angles: 8.069±0.200°, 11.606±0.200°, 13.120±0.200°, and 19.812±0.200°;

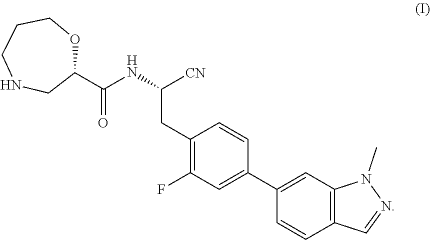 [SMILES: Cn1ncc2ccc(-c3ccc(C[C@@H](C#N)NC(=O)[C@@H]4CNCCCO4)c(F)c3)cc21]

In some embodiments of the present invention, the X-ray powder diffraction pattern of the above crystalline form C has characteristic diffraction peaks at the following 2θ angles: 8.069±0.200°, 11.606±0.200°, 13.120±0.200°, 13.988±0.200°, 15.873±0.200°, 19.812±0.200°, and 22.076±0.200°.

In some embodiments of the present invention, the X-ray powder diffraction pattern of the above crystalline form C has characteristic diffraction peaks at the following 2θ angles: 8.069±0.200°, 9.557±0.200°, 11.606±0.200°, 13.120±0.200°, 13.988±0.200°, 15.873±0.200°, 16.314±0.200°, 19.812±0.200°, 20.416±0.200°, and 22.076±0.200°.

In some embodiments of the present invention, the X-ray powder diffraction pattern of the above crystalline form C has characteristic diffraction peaks at the following 2θ angles: 6.478±0.200°, 8.069±0.200°, 9.557±0.200°, 11.606±0.200°, 13.120±0.200°, 13.988±0.200°, 15.873±0.200°, 16.314±0.200°, 17.312±0.200°, 17.900±0.200°, 18.287±0.200°, 19.349±0.200°, 19.812±0.200°, 20.416±0.200°, 21.116±0.200°, 22.076±0.200°, 22.673±0.200°, 23.464±0.200°, 23.807±0.200°, 24.271±0.200°, 24.637±0.200°, 26.569±0.200°, 26.988±0.200°, 28.326±0.200°, 30.416±0.200°, and 33.799±0.200°.

In some embodiments of the present invention, the X-ray powder diffraction pattern of the above crystalline form C has characteristic diffraction peaks at the following 2θ angles: 6.478±0.100°, 8.069±0.100°, 9.557±0.100°, 11.606±0.100°, 13.120±0.100°, 13.988±0.100°, 15.873±0.100°, 16.314±0.100°, 17.312±0.100°, 17.900±0.100°, 18.287±0.100°, 19.349±0.100°, 19.812±0.100°, 20.416±0.100°, 21.116±0.100°, 22.076±0.100°, 22.673±0.100°, 23.464±0.100°, 23.807±0.100°, 24.271±0.100°, 24.637±0.100°, 26.569±0.100°, 26.988±0.100°, 28.326±0.100°, 30.416±0.100°, and 33.799±0.100°.

In some embodiments of the present invention, the X-ray powder diffraction pattern of the above crystalline form C has characteristic diffraction peaks at the following 2θ angles: 8.069±0.200°, 11.606±0.200°, 13.120±0.200°, and/or 6.478±0.200°, and/or 9.557±0.200°, and/or 13.988±0.200°, and/or 15.873±0.200°, and/or 16.314±0.200°, and/or 17.312±0.200°, and/or 17.900±0.200°, and/or 18.287±0.200°, and/or 19.349±0.200°, and/or 19.812±0.200°, and/or 20.416±0.200°, and/or 21.116±0.200°, and/or 22.076±0.200°, and/or 22.673±0.200°, and/or 23.464±0.200°, and/or 23.807±0.200°, and/or 24.271±0.200°, and/or 24.637±0.200°, and/or 26.569±0.200°, and/or 26.988±0.200°, and/or 28.326±0.200°, and/or 30.416±0.200°, and/or 33.799±0.200°.

In some embodiments of the present invention, the X-ray powder diffraction pattern of the above crystalline form C has characteristic diffraction peaks at the following 2θ angles: 8.069±0.100°, 11.606±0.100°, 13.120±0.100°, and/or 6.478±0.100°, and/or 9.557±0.100°, and/or 13.988±0.100°, and/or 15.873±0.100°, and/or 16.314±0.100°, and/or 17.312±0.100°, and/or 17.900±0.100°, and/or 18.287±0.100°, and/or 19.349±0.100°, and/or 19.812±0.100°, and/or 20.416±0.100°, and/or 21.116±0.100°, and/or 22.076±0.100°, and/or 22.673±0.100°, and/or 23.464±0.100°, and/or 23.807±0.100°, and/or 24.271±0.100°, and/or 24.637±0.100°, and/or 26.569±0.100°, and/or 26.988±0.100°, and/or 28.326±0.100°, and/or 30.416±0.100°, and/or 33.799±0.100°.

In some embodiments of the present invention, the X-ray powder diffraction pattern of the above crystalline form C has characteristic diffraction peaks at the following 2θ angles: 6.478°, 8.069°, 9.557°, 11.606°, 13.120°, 13.988°, 15.873°, 16.314°, 17.312°, 17.900°, 18.287°, 19.349°, 19.812°, 20.416°, 21.116°, 22.076°, 22.673°, 23.464°, 23.807°, 24.271°, 24.637°, 26.569°, 26.988°, 28.326°, 30.416°, and 33.799°.

In some embodiments of the present invention, the XRPD pattern analysis data of the above crystalline form C is as shown in Table 3.

TABLE 3
XRPD analysis data of the crystalline
form C of the compound of formula (I)

| | 2θ | Peak | | Relative |
|---|---|---|---|---|
| | angle | height | Interplanar | intensity |
| No. | [°2θ] | [cts] | spacing (Å) | (%) |
| 1 | 6.478 | 148.664 | 13.63300 | 5.5 |
| 2 | 8.069 | 695.194 | 10.94793 | 41.1 |
| 3 | 9.557 | 344.083 | 9.24674 | 18.5 |
| 4 | 11.606 | 731.602 | 7.61885 | 43.3 |
| 5 | 13.120 | 1617.10 | 6.74249 | 100.0 |
| 6 | 13.988 | 570.057 | 6.32632 | 31.8 |
| 7 | 15.873 | 657.462 | 5.57875 | 37.8 |
| 8 | 16.314 | 270.203 | 5.42907 | 13.0 |
| 9 | 17.312 | 99.4029 | 5.11832 | 2.2 |
| 10 | 17.900 | 94.2935 | 4.95153 | 1.8 |
| 11 | 18.287 | 92.9355 | 4.84744 | 1.8 |
| 12 | 19.349 | 148.959 | 4.58368 | 5.4 |
| 13 | 19.812 | 1049.33 | 4.47767 | 63.4 |
| 14 | 20.416 | 355.770 | 4.34653 | 18.3 |
| 15 | 21.116 | 147.101 | 4.20393 | 4.7 |
| 16 | 22.076 | 712.163 | 4.02338 | 41.4 |
| 17 | 22.673 | 121.040 | 3.91866 | 3.3 |
| 18 | 23.464 | 91.3495 | 3.78828 | 1.8 |
| 19 | 23.807 | 109.263 | 3.73450 | 3.1 |
| 20 | 24.271 | 152.001 | 3.66423 | 6.1 |
| 21 | 24.637 | 105.693 | 3.61057 | 3.4 |
| 22 | 26.569 | 159.844 | 3.35227 | 6.8 |
| 23 | 26.988 | 115.966 | 3.30111 | 4.0 |
| 24 | 28.326 | 98.3310 | 3.14821 | 3.3 |
| 25 | 30.416 | 138.068 | 2.93642 | 6.2 |
| 26 | 33.799 | 82.0005 | 2.64984 | 3.1 |

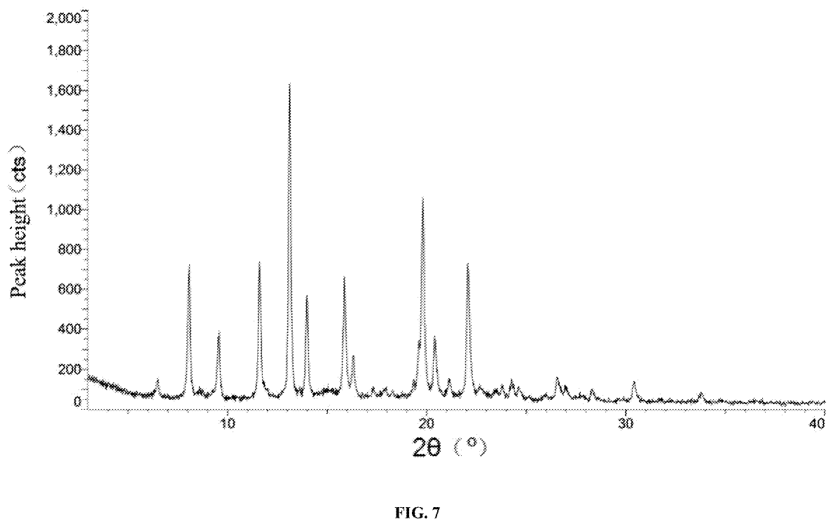

In some embodiments of the present invention, the XRPD pattern of the above crystalline form C is substantially as shown in FIG. 7.

In some embodiments of the present invention, the differential scanning calorimetry curve of the above crystalline form C has peak values of endothermic peaks at 78.70±3° C. and 140.29±3° C.

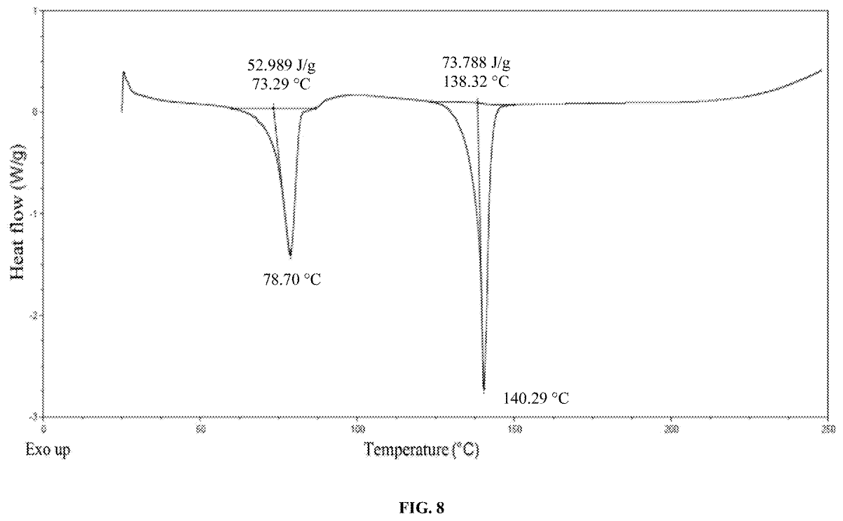

In some embodiments of the present invention, the DSC pattern of the above crystalline form C is substantially as shown in FIG. 8.

In some embodiments of the present invention, the thermogravimetric analysis curve of the above crystalline form C shows a weight loss of 6.495% at 100.00±3° C.

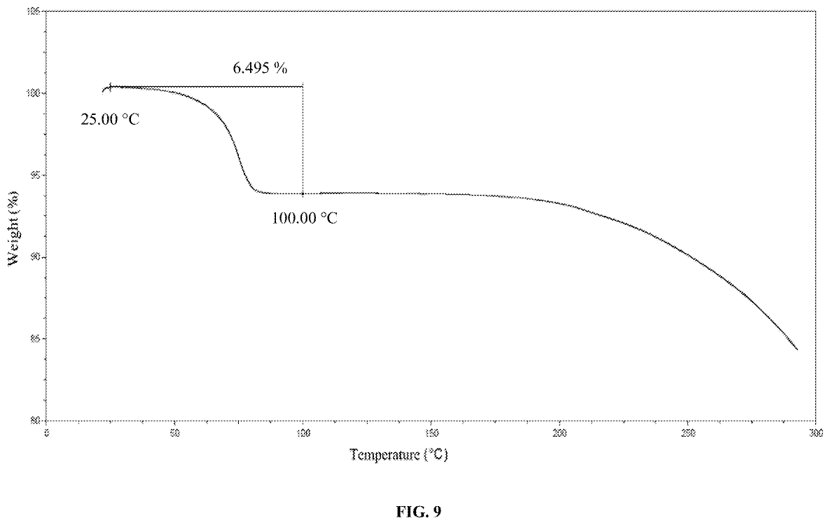

In some embodiments of the present invention, the TGA pattern of the above crystalline form C is substantially as shown in FIG. 9.

In some embodiments of the present invention, the above crystalline form C is an ethanol solvate crystalline form.

The present invention further provides the crystalline form D of the compound of formula (I), and its X-ray powder diffraction pattern has characteristic diffraction peaks at the following 2θ angles: 9.105±0.200°, 15.206±0.200°, and 16.904±0.200°;

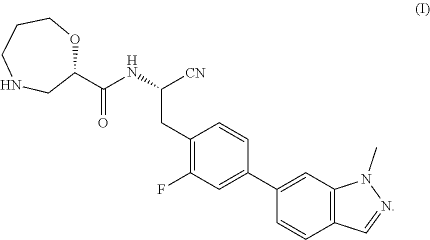 [SMILES: Cn1ncc2ccc(-c3ccc(C[C@@H](C#N)NC(=O)[C@@H]4CNCCCO4)c(F)c3)cc21]

In some embodiments of the present invention, the X-ray powder diffraction pattern of the above crystalline form D has characteristic diffraction peaks at the following 2θ angles: 9.105±0.200°, 9.751±0.200°, 13.745±0.200°, 15.206±0.200°, 16.904±0.200°, and 18.075±0.200°.

In some embodiments of the present invention, the X-ray powder diffraction pattern of the above crystalline form D has characteristic diffraction peaks at the following 2θ angles: 9.105±0.200°, 9.751±0.200°, 13.745±0.200°, 14.031±0.200°, 15.206±0.200°, 16.904±0.200°, 17.316±0.200°, 18.075±0.200°, 18.381±0.200°, 22.434±0.200°, and 22.659±0.200°.

In some embodiments of the present invention, the X-ray powder diffraction pattern of the above crystalline form D has characteristic diffraction peaks at the following 2θ angles: 9.105±0.100°, 9.751±0.100°, 13.745±0.100°, 14.031±0.100°, 15.206±0.100°, 16.904±0.100°, 17.316±0.100°, 18.075±0.100°, 18.381±0.100°, 22.434±0.100°, and 22.659±0.100°.

In some embodiments of the present invention, the X-ray powder diffraction pattern of the above crystalline form D has characteristic diffraction peaks at the following 2θ angles: 9.105±0.200°, 9.751±0.200°, 11.830±0.200°, 13.745±0.200°, 14.031±0.200°, 15.206±0.200°, 16.171±0.200°, 16.904±0.200°, 17.316±0.200°, 18.075±0.200°, 18.381±0.200°, 18.825±0.200°, 22.434±0.200°, 22.659±0.200°, and 27.269±0.200°.

In some embodiments of the present invention, the X-ray powder diffraction pattern of the above crystalline form D has characteristic diffraction peaks at the following 2θ angles: 9.105±0.100°, 9.751±0.100°, 11.830±0.100°, 13.745±0.100°, 14.031±0.100°, 15.206±0.100°, 16.171±0.100°, 16.904±0.100°, 17.316±0.100°, 18.075±0.100°, 18.381±0.100°, 18.825±0.100°, 22.434±0.100°, 22.659±0.100°, and 27.269±0.100°.

In some embodiments of the present invention, the X-ray powder diffraction pattern of the above crystalline form D has characteristic diffraction peaks at the following 2θ angles: 4.477±0.200°, 7.374±0.200°, 8.218±0.200°, 9.105±0.200°, 9.751±0.200°, 10.371±0.200°, 11.830±0.200°, 12.113±0.200°, 13.351±0.200°, 13.745±0.200°, 14.031±0.200°, 15.206±0.200°, 15.361±0.200°, 16.171±0.200°, 16.672±0.200°, 16.904±0.200°, 17.316±0.200°, 18.075±0.200°, 18.381±0.200°, 18.825±0.200°, 19.690±0.200°, 20.141±0.200°, 20.449±0.200°, 21.349±0.200°, 22.434±0.200°, 22.659±0.200°, 23.294±0.200°, 23.692±0.200°, 24.482±0.200°, 25.049±0.200°, 25.805±0.200°, 26.062±0.200°, 26.812±0.200°, 27.269±0.200°, 28.229±0.200°, 28.586±0.200°, 30.933±0.200°, 32.633±0.200°, 33.574±0.200°, 34.384±0.200°, and 39.672±0.200°.

In some embodiments of the present invention, the X-ray powder diffraction pattern of the above crystalline form D has characteristic diffraction peaks at the following 2θ angles: 4.477±0.100°, 7.374±0.100°, 8.218±0.100°, 9.105±0.100°, 9.751±0.100°, 10.371±0.100°, 11.830±0.100°, 12.113±0.100°, 13.351±0.100°, 13.745±0.100°, 14.031±0.100°, 15.206±0.100°, 15.361±0.100°, 16.171±0.100°, 16.672±0.100°, 16.904±0.100°, 17.316±0.100°, 18.075±0.100°, 18.381±0.100°, 18.825±0.100°, 19.690±0.100°, 20.141±0.100°, 20.449±0.100°, 21.349±0.100°, 22.434±0.100°, 22.659±0.100°, 23.294±0.100°, 23.692±0.100°, 24.482±0.100°, 25.049±0.100°, 25.805±0.100°, 26.062±0.100°, 26.812±0.100°, 27.269±0.100°, 28.229±0.100°, 28.586±0.100°, 30.933±0.100°, 32.633±0.100°, 33.574±0.100°, 34.384±0.100°, and 39.672±0.100°.

In some embodiments of the present invention, the X-ray powder diffraction pattern of the above crystalline form D has characteristic diffraction peaks at the following 2θ angles: 8.069±0.200°, 11.606±0.200°, 13.120±0.200°, and/or 6.478±0.200°, and/or 9.557±0.200°, and/or 13.988±0.200°, and/or 15.873±0.200°, and/or 16.314±0.200°, and/or 17.312±0.200°, and/or 17.900±0.200°, and/or 18.287±0.200°, and/or 19.349±0.200°, and/or 19.812±0.200°, and/or 20.416±0.200°, and/or 21.116±0.200°, and/or 22.076±0.200°, and/or 22.673±0.200°, and/or 23.464±0.200°, and/or 23.807±0.200°, and/or 24.271±0.200°, and/or 24.637±0.200°, and/or 26.569±0.200°, and/or 26.988±0.200°, and/or 28.326±0.200°, and/or 30.416±0.200°, and/or 33.799±0.200°.

In some embodiments of the present invention, the X-ray powder diffraction pattern of the above crystalline form D has characteristic diffraction peaks at the following 2θ angles: 8.069±0.100°, 11.606±0.100°, 13.120±0.100°, and/or 6.478±0.100°, and/or 9.557±0.100°, and/or 13.988±0.100°, and/or 15.873±0.100°, and/or 16.314±0.100°, and/or 17.312±0.100°, and/or 17.900±0.100°, and/or 18.287±0.100°, and/or 19.349±0.100°, and/or 19.812±0.100°, and/or 20.416±0.100°, and/or 21.116±0.100°, and/or 22.076±0.100°, and/or 22.673±0.100°, and/or 23.464±0.100°, and/or 23.807±0.100°, and/or 24.271±0.100°, and/or 24.637±0.100°, and/or 26.569±0.100°, and/or 26.988±0.100°, and/or 28.326±0.100°, and/or 30.416±0.100°, and/or 33.799±0.100°.

In some embodiments of the present invention, the X-ray powder diffraction pattern of the above crystalline form D has characteristic diffraction peaks at the following 2θ angles: 4477°, 7.374°, 8.218°, 9.105°, 9.751°, 10.371°, 11.83°, 12.113°, 13.351°, 13.745°, 14.031°, 15.206°, 15.361°, 16.171°, 16.672°, 16.904°, 17.316°, 18.075°, 18.381°, 18.825°, 19.690°, 20.141°, 20.449°, 21.349°, 22.434°, 22.659°, 23.294°, 23.692°, 24.482°, 25.049°, 25.805°, 26.062°, 26.812°, 27.269°, 28.229°, 28.586°, 30.933°, 32.633°, 33.574°, 34.384°, and 39.672°.

In some embodiments of the present invention, the XRPD pattern analysis data of the above crystalline form D is as shown in Table 4.

TABLE 4
XRPD analysis data of the crystalline
form D of the compound of formula (I)

| | 2θ | Peak | Interplanar | Relative |
|---|---|---|---|---|
| | angle | height | spacing | intensity |
| No. | [°2θ] | [cts] | (Å) | (%) |
| 1 | 4.477 | 230.827 | 19.72171 | 14.6 |
| 2 | 7.374 | 99.9114 | 11.97872 | 4.0 |
| 3 | 8.218 | 178.828 | 10.74978 | 12.1 |
| 4 | 9.105 | 978.653 | 9.70494 | 100.0 |
| 5 | 9.751 | 276.214 | 9.06330 | 22.3 |
| 6 | 10.371 | 162.748 | 8.52291 | 10.1 |
| 7 | 11.830 | 218.796 | 7.47493 | 15.3 |
| 8 | 12.113 | 178.727 | 7.30078 | 10.7 |
| 9 | 13.351 | 201.501 | 6.62651 | 11.6 |
| 10 | 13.745 | 316.758 | 6.43726 | 23.5 |
| 11 | 14.031 | 315.028 | 6.30678 | 22.8 |
| 12 | 15.206 | 966.167 | 5.82216 | 92.9 |
| 13 | 15.361 | 380.620 | 5.76373 | 28.0 |
| 14 | 16.171 | 301.262 | 5.47654 | 18.5 |
| 15 | 16.672 | 260.723 | 5.31333 | 13.7 |
| 16 | 16.904 | 691.577 | 5.24094 | 61.3 |
| 17 | 17.316 | 336.172 | 5.11698 | 22.0 |
| 18 | 18.075 | 378.781 | 4.90389 | 27.0 |
| 19 | 18.381 | 354.220 | 4.82286 | 24.5 |
| 20 | 18.825 | 285.661 | 4.71018 | 17.4 |
| 21 | 19.690 | 236.087 | 4.50505 | 11.8 |
| 22 | 20.141 | 353.034 | 4.40533 | 24.6 |
| 23 | 20.449 | 327.348 | 4.33965 | 21.7 |
| 24 | 21.349 | 184.629 | 4.15867 | 6.1 |
| 25 | 22.434 | 398.512 | 3.95986 | 29.3 |
| 26 | 22.659 | 409.091 | 3.92108 | 30.3 |
| 27 | 23.294 | 183.402 | 3.81566 | 4.9 |
| 28 | 23.692 | 260.644 | 3.75240 | 13.3 |
| 29 | 24.482 | 241.114 | 3.63310 | 11.2 |
| 30 | 25.049 | 285.316 | 3.55206 | 16.4 |
| 31 | 25.805 | 230.322 | 3.44967 | 11.1 |
| 32 | 26.062 | 295.526 | 3.41625 | 18.6 |
| 33 | 26.812 | 164.402 | 3.32242 | 5.4 |
| 34 | 27.269 | 308.476 | 3.26771 | 22.3 |
| 35 | 28.229 | 154.169 | 3.15877 | 6.1 |
| 36 | 28.586 | 199.402 | 3.12013 | 11.1 |
| 37 | 30.933 | 132.052 | 2.88857 | 5.8 |
| 38 | 32.633 | 112.000 | 2.74184 | 4.6 |
| 39 | 33.574 | 93.0000 | 2.66713 | 2.8 |
| 40 | 34.384 | 103.322 | 2.60608 | 4.4 |
| 41 | 39.672 | 77.1068 | 2.27009 | 2.6 |

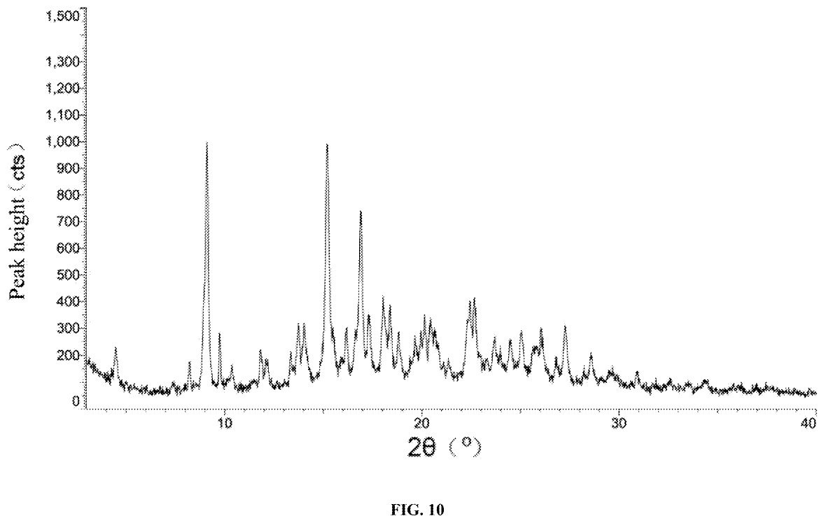

In some embodiments of the present invention, the XRPD pattern of the above crystalline form D is substantially as shown in FIG. 10.

In some embodiments of the present invention, the differential scanning calorimetry curve of the above crystalline form D has peak values of endothermic peaks at 88.63±3° C. and 140.53±3° C.

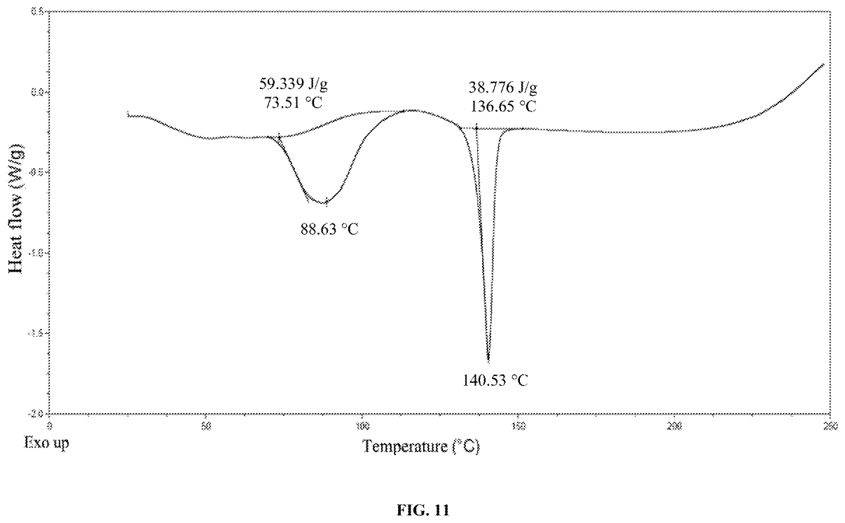

In some embodiments of the present invention, the DSC pattern of the above crystalline form D is substantially as shown in FIG. 11.

In some embodiments of the present invention, the thermogravimetric analysis curve of the above crystalline form D shows a weight loss of 2.273% at 120.00±3° C.

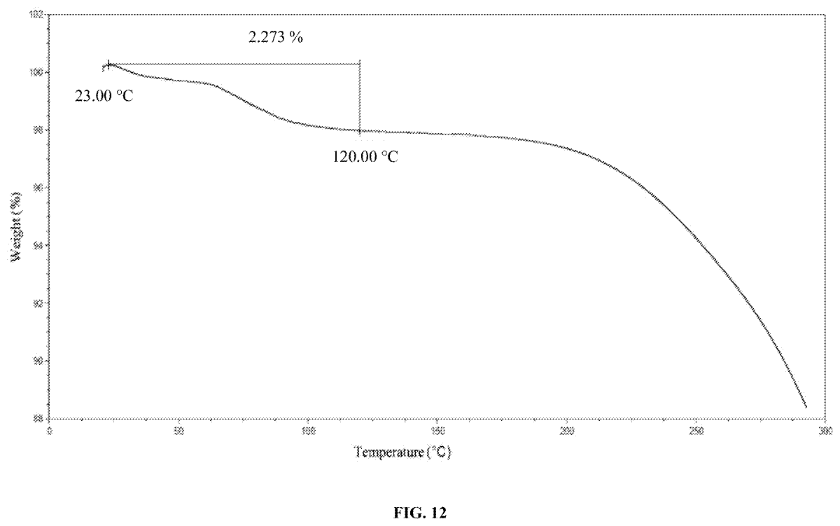

In some embodiments of the present invention, the TGA pattern of the above crystalline form D is substantially as shown in FIG. 12.

In some embodiments of the present invention, the above crystalline form D is a hydrate.

The present invention further provides the crystalline form E of the compound of formula (I), and its X-ray powder diffraction pattern has characteristic diffraction peaks at the following 2θ angles: 9.331±0.200°, 12.051±0.200°, and 15.214±0.200°;

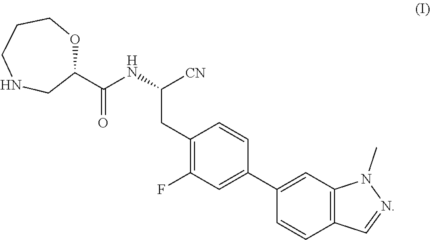 [SMILES: Cn1ncc2ccc(-c3ccc(C[C@@H](C#N)NC(=O)[C@@H]4CNCCCO4)c(F)c3)cc21]

In some embodiments of the present invention, the X-ray powder diffraction pattern of the above crystalline form E has characteristic diffraction peaks at the following 2θ angles: 9.331±0.200°, 12.051±0.200°, 15.214±0.200°, 15.988±0.200°, 16.749±0.200°.

In some embodiments of the present invention, the X-ray powder diffraction pattern of the above crystalline form E has characteristic diffraction peaks at the following 2θ angles: 9.331±0.200°, 12.051±0.200°, 15.214±0.200°, 15.988±0.200°, 16.749±0.200°, 20.522±0.200°.

In some embodiments of the present invention, the X-ray powder diffraction pattern of the above crystalline form E has characteristic diffraction peaks at the following 2θ angles: 9.331±0.200°, 12.051±0.200°, 13.671±0.200°, 15.214±0.200°, 15.988±0.200°, 16.749±0.200°, 17.932±0.200°, 20.522±0.200°.

In some embodiments of the present invention, the X-ray powder diffraction pattern of the above crystalline form E has characteristic diffraction peaks at the following 2θ angles: 8.876±0.200°, 9.331±0.200°, 10.126±0.200°, 12.051±0.200°, 13.671±0.200°, 15.214±0.200°, 15.988±0.200°, 16.749±0.200°, 17.932±0.200°, 20.522±0.200°, 22.933±0.200°, 23.443±0.200°.

In some embodiments of the present invention, the X-ray powder diffraction pattern of the above crystalline form E has characteristic diffraction peaks at the following 2θ angles: 7.539±0.200°, 8.876±0.200°, 9.331±0.200°, 10.126±0.200°, 11.566±0.200°, 12.051±0.200°, 13.169±0.200°, 13.671±0.200°, 15.214±0.200°, 15.988±0.200°, 16.749±0.200°, 17.398±0.200°, 17.932±0.200°, 18.823±0.200°, 19.722±0.200°, 20.522±0.200°, 20.819±0.200°, 21.288±0.200°, 22.933±0.200°, 23.443±0.200°, 24.005±0.200°, 24.364±0.200°, 24.861±0.200°, 26.357±0.200°, 27.064±0.200°, 27.606±0.200°, 28.189±0.200°, 29.117±0.200°, 29.932±0.200°, 30.932±0.200°, 32.265±0.200°, 33.273±0.200°, and 34.094±0.200°.

In some embodiments of the present invention, the X-ray powder diffraction pattern of the above crystalline form E has characteristic diffraction peaks at the following 2θ angles: 7.539±0.100°, 8.876±0.100°, 9.331±0.100°, 10.126±0.100°, 11.566±0.100°, 12.051±0.100°, 13.169±0.100°, 13.671±0.100°, 15.214±0.100°, 15.988±0.100°, 16.749±0.100°, 17.398±0.100°, 17.932±0.100°, 18.823±0.100°, 19.722±0.100°, 20.522±0.100°, 20.819±0.100°, 21.288±0.100°, 22.933±0.100°, 23.443±0.100°, 24.005±0.100°, 24.364±0.100°, 24.861±0.100°, 26.357±0.100°, 27.064±0.100°, 27.606±0.100°, 28.189±0.100°, 29.117±0.100°, 29.932±0.100°, 30.932±0.100°, 32.265±0.100°, 33.273±0.100°, and 34.094±0.100°.

In some embodiments of the present invention, the X-ray powder diffraction pattern of the above crystalline form E has characteristic diffraction peaks at the following 2θ angles: 9.331±0.200°, 12.051±0.200°, 15.214±0.200°, and/or 7.539±0.200°, and/or 8.876±0.200°, and/or 10.126±0.200°, and/or 11.566±0.200°, and/or 13.169±0.200°, and/or 13.671±0.200°, and/or 15.988±0.200°, and/or 16.749±0.200°, and/or 17.398±0.200°, and/or 17.932±0.200°, and/or 18.823±0.200°, and/or 19.722±0.200°, and/or 20.522±0.200°, and/or 20.819±0.200°, and/or 21.288±0.200°, and/or 22.933±0.200°, and/or 23.443±0.200°, and/or 24.005±0.200°, and/or 24.364±0.200°, and/or 24.861±0.200°, and/or 26.357±0.200°, and/or 27.064±0.200°, and/or 27.606±0.200°, and/or 28.189±0.200°, and/or 29.117±0.200°, and/or 29.932±0.200°, and/or 30.932±0.200°, and/or 32.265±0.200°, and/or 33.273±0.200°, and/or 34.094±0.200°.

In some embodiments of the present invention, the X-ray powder diffraction pattern of the above crystalline form E has characteristic diffraction peaks at the following 2θ angles: 9.331±0.100°, 12.051±0.100°, 15.214±0.100°, and/or 7.539±0.100°, and/or 8.876±0.100°, and/or 10.126±0.100°, and/or 11.566±0.100°, and/or 13.169±0.100°, and/or 13.671±0.100°, and/or 15.988±0.100°, and/or 16.749±0.100°, and/or 17.398±0.100°, and/or 17.932±0.100°, and/or 18.823±0.100°, and/or 19.722±0.100°, and/or 20.522±0.100°, and/or 20.819±0.100°, and/or 21.288±0.100°, and/or 22.933±0.100°, and/or 23.443±0.100°, and/or 24.005±0.100°, and/or 24.364±0.100°, and/or 24.861±0.100°, and/or 26.357±0.100°, and/or 27.064±0.100°, and/or 27.606±0.100°, and/or 28.189±0.100°, and/or 29.117±0.100°, and/or 29.932±0.100°, and/or 30.932±0.100°, and/or 32.265±0.100°, and/or 33.273±0.100°, and/or 34.094±0.100°.

In some embodiments of the present invention, the X-ray powder diffraction pattern of the above crystalline form E has characteristic diffraction peaks at the following 2θ angles: 7.539°, 8.876°, 9.331°, 10.126°, 11.566°, 12.051°, 13.169°, 13.671°, 15.214°, 15.988°, 16.749°, 17.398°, 17.932°, 18.823°, 19.722°, 20.522°, 20.819°, 21.288°, 22.933°, 23.443°, 24.005°, 24.364°, 24.861°, 26.357°, 27.064°, 27.606°, 28.189°, 29.117°, 29.932°, 30.932°, 32.265°, 33.273°, and 34.094°.

In some embodiments of the present invention, the XRPD pattern analysis data of the above crystalline form E is as shown in Table 5.

TABLE 5
XRPD analysis data of the crystalline
form E of the compound of formula (I)

| | 2θ | Peak | | Relative |
|---|---|---|---|---|
| | angle | height | Interplanar | intensity |
| No. | [°2θ] | [cts] | spacing (Å) | (%) |
| 1 | 7.539 | 119.118 | 11.71722 | 5.3 |
| 2 | 8.876 | 378.292 | 9.95460 | 27.8 |
| 3 | 9.331 | 1058.29 | 9.47012 | 87.9 |
| 4 | 10.126 | 237.293 | 8.72840 | 15.6 |
| 5 | 11.566 | 171.000 | 7.64489 | 9.9 |
| 6 | 12.051 | 1190.00 | 7.33805 | 100.0 |
| 7 | 13.169 | 101.363 | 6.71742 | 3.9 |
| 8 | 13.671 | 443.051 | 6.47222 | 34.2 |
| 9 | 15.214 | 843.311 | 5.81891 | 69.0 |
| 10 | 15.988 | 613.100 | 5.53880 | 48.0 |
| 11 | 16.749 | 638.337 | 5.28891 | 50.0 |
| 12 | 17.398 | 194.002 | 5.09306 | 10.8 |
| 13 | 17.932 | 523.960 | 4.94253 | 40.2 |
| 14 | 18.823 | 171.197 | 4.71074 | 9.8 |
| 15 | 19.722 | 185.600 | 4.49782 | 11.4 |
| 16 | 20.522 | 529.232 | 4.32435 | 41.6 |
| 17 | 20.819 | 223.654 | 4.26327 | 14.5 |
| 18 | 21.288 | 117.083 | 4.17038 | 5.2 |
| 19 | 22.933 | 251.700 | 3.87481 | 16.6 |
| 20 | 23.443 | 330.635 | 3.79162 | 23.4 |
| 21 | 24.005 | 158.445 | 3.70418 | 8.3 |
| 22 | 24.364 | 147.250 | 3.65037 | 7.4 |
| 23 | 24.861 | 108.043 | 3.57849 | 4.2 |
| 24 | 26.357 | 136.501 | 3.37877 | 7.3 |
| 25 | 27.064 | 155.000 | 3.29207 | 9.4 |
| 26 | 27.606 | 85.5000 | 3.22857 | 3.8 |
| 27 | 28.189 | 58.0161 | 3.16320 | 1.8 |
| 28 | 29.117 | 56.3642 | 3.06447 | 1.8 |
| 29 | 29.932 | 90.0715 | 2.98281 | 4.6 |
| 30 | 30.932 | 63.5615 | 2.88859 | 2.1 |
| 31 | 32.265 | 85.7000 | 2.77229 | 4.2 |
| 32 | 33.273 | 69.7739 | 2.69052 | 2.9 |
| 33 | 34.094 | 72.7078 | 2.62762 | 3.2 |

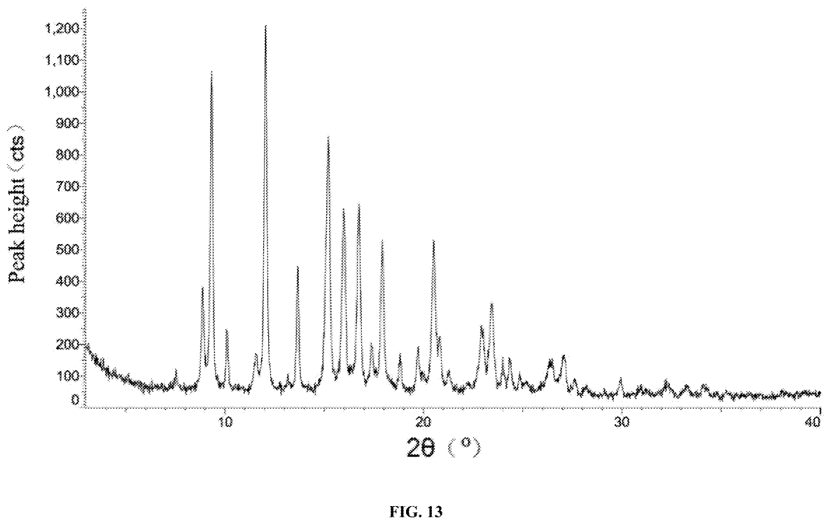

In some embodiments of the present invention, the XRPD pattern of the above crystalline form E of the compound of formula (I) is substantially as shown in FIG. 13.

In some embodiments of the present invention, the differential scanning calorimetry curve of the above crystalline form E of the compound of formula (I) has a peak value of an exothermic peak at 96.78±3° C. and a peak value of an endothermic peak at 140.12±3° C.

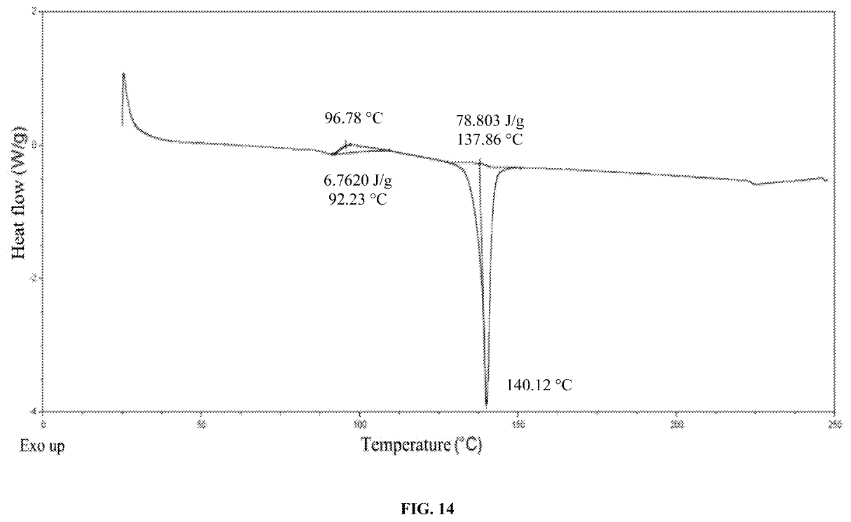

In some embodiments of the present invention, the DSC pattern of the above crystalline form E of the compound of formula (I) is substantially as shown in FIG. 14.

In some embodiments of the present invention, the thermogravimetric analysis curve of the above crystalline form E of the compound of formula (I) shows a weight loss of 0.293% at 100.00±3° C.

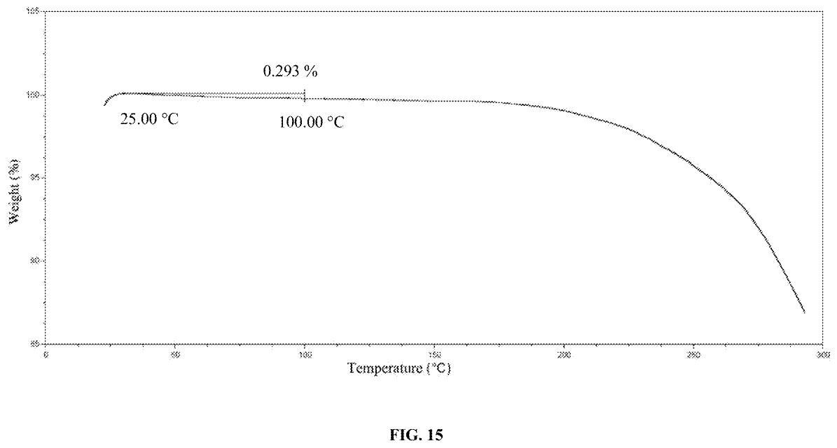

In some embodiments of the present invention, the TGA pattern of the above crystalline form E of the compound of formula (I) is substantially as shown in FIG. 15.

In some embodiments of the present invention, the above crystalline form E of the compound of formula (I) is an anhydrous form.

In a second aspect, the present invention provides a pharmaceutical composition comprising a crystalline form according to any one or more crystalline forms of the present invention, and a pharmaceutically acceptable carrier.

In some embodiments of the present invention, the crystalline form is crystalline form A.

In another aspect, the present invention further provides use of an above crystalline form of the compound of formula (I) in the preparation of a DPP1 inhibitor.

In another aspect, the present invention further provides use of the above crystalline form A of the compound of formula (I) in the preparation of a DPP1 inhibitor.

In another aspect, the present invention further provides use of an above crystalline form of the compound of formula (I) in the preparation of a medicament for preventing or treating a DPP1-related disease.

In some embodiments of the present invention, the DPP1-related disease is chronic obstructive pulmonary disease.

In another aspect, the present invention further provides use of the above crystalline form A of the compound of formula (I) in the preparation of a medicament for preventing or treating a DPP1-related disease.

In another aspect, the present invention further provides a method for preventing or treating a DPP1-related disease in a subject in need thereof, comprising providing to the subject an effective dose of an above crystalline form of the compound of formula (I).

In another aspect, the present invention further provides a method for preventing or treating a DPP1-related disease in a subject in need thereof, comprising providing to the subject an effective dose of the above crystalline form A of the compound of formula (I).

In another aspect, the present invention further provides use of an above crystalline form of the compound of formula (I) in the prevention or treatment of a DPP1-related disease.

In another aspect, the present invention further provides the above crystalline form A of the compound of formula (I) for preventing or treating a DPP1-related disease.

In another aspect, the present invention further provides a method for preparing the crystalline form A of the compound of formula (I), comprising the steps of:
stirring the compound of formula (I) in a mixed solvent of isopropanol and water until a solid precipitates, filtering the solid, and drying the solid at 20-45° C. (preferably 30-40° C.) to obtain the crystalline form A.

In another preferred embodiment, the stirring is carried out at 40-55° C. (preferably 45-50° C.) for 1-6 h (preferably 2-4 h), followed by cooling to 15-30° C. (preferably 20-25° C.) and stirring for 0.5-3 h (preferably 1-2 h).

In another preferred embodiment, the drying is vacuum drying.

In another preferred embodiment, in the mixed solvent, the volume ratio of isopropanol to water is 10-20:1, preferably 14-16:1.

In another preferred embodiment, the mass-to-volume ratio of the compound of formula (I) to the mixed solvent is 20-100 g/L, preferably 30-80 g/L, and more preferably 50-70 g/L.

In another aspect, the present invention further provides a method for preparing the crystalline form B of the compound of formula (I), comprising the steps of:
stirring the compound of formula (I) in ethyl acetate until a solid precipitates, filtering the solid, and purging the obtained solid with nitrogen (for 2-6 h) to obtain the crystalline form B.

In another preferred embodiment, the stirring is carried out at 40-55° C. (preferably 45-50° C.) for 1-5 days (preferably 2-4 days).

In another preferred embodiment, the mass-to-volume ratio of the compound of formula (I) to ethyl acetate is 20-80 g/L, preferably 30-60 g/L, and more preferably 40-50 g/L.

In another aspect, the present invention further provides a method for preparing the crystalline form C of the compound of formula (I), comprising the steps of:
providing an ethanol solution of the compound of formula (I), cooling the solution to −8 to −20° C. (preferably −10 to −15° C.) and stirring the solution until a solid precipitates, filtering the solid, and purging the obtained solid with nitrogen (for 2-6 h) to obtain the crystalline form C.

In another preferred embodiment, the stirring is carried out for 10-30 h, preferably 16-24 h.

In another preferred embodiment, the mass-to-volume ratio of the compound of formula (I) to ethanol is 20-80 g/L, preferably 30-60 g/L, and more preferably 40-50 g/L.

In another aspect, the present invention further provides a method for preparing the crystalline form D of the compound of formula (I), comprising the steps of:
providing a methanol solution of the compound of formula (I), then allowing the solution to evaporate slowly at 15-30° C. (preferably 18-22° C.) to obtain the crystalline form D.

In another preferred embodiment, the mass-to-volume ratio of the compound of formula (I) to methanol is 10-80 g/L, preferably 20-60 g/L, and more preferably 30-40 g/L.

In another aspect, the present invention further provides a method for preparing the crystalline form E of the compound of formula (I), comprising the steps of:
providing an ethanol solution of the compound of formula (I), cooling the solution to −8 to −20° C. (preferably −10 to −15° C.) until a solid precipitates, filtering the solid, and drying the obtained solid at 40-55° C. (preferably 30-40° C.) to obtain the crystalline form E.

In another preferred embodiment, the stirring is carried out for 10-30 h, preferably 16-24 h.

In another preferred embodiment, the mass-to-volume ratio of the compound of formula (I) to ethanol is 20-80 g/L, preferably 20-60 g/L, and more preferably 30-40 g/L.

A method for preparing the crystalline form E, comprising the step of:
drying the crystalline form C at 40-55° C. (preferably 30-40° C.) to obtain the crystalline form E. It should be understood that, within the scope of the present invention, each of the above-mentioned technical features of the present invention and the technical features specifically described hereinafter (such as in Examples) can be combined with each other to form new or preferred technical solutions. Due to space limitations, such combinations will not be described individually herein.

### Certain Terminology

The section headings used herein are for organizational purposes only and are not to be construed as limiting the subject matter described.

Unless otherwise stated, the following terms used in this application have the definitions given below.

Unless otherwise defined, all technical and scientific terms used herein have the same meanings as those commonly understood by a person of ordinary skill in the art to which the present invention belongs.

As used herein, the terms “contain”, “include”, “includes,” or “included” is not limiting.

As used herein, the term “room temperature” or “normal temperature” refers to a temperature of 4-40° C., preferably 25±5° C.

Unless otherwise specified, X-ray powder diffraction (XRPD) can be used to detect changes in crystals, degree of crystallinity, crystalline structure state, and other information, and is a commonly used means for identification of crystals. The peak positions of an XRPD pattern are primarily determined by the structure of a crystal and are relatively insensitive to experimental details, whereas the relative peak heights depend on many factors related to sample preparation and instrument geometry. Therefore, for any given crystalline form, the relative intensity of diffraction peaks may vary due to preferred orientation arising from factors such as crystalline morphology, as is well known in the field of crystallography. In the presence of preferred orientation effects, the peak intensity varies, but the positions of diffraction peaks of a crystal remain unchanged. In addition, for any given crystal, there may be slight errors in the position of a peak, which is also well known in the field of crystallography. For example, due to factors such as temperature changes, sample movement, or instrument calibration during sample analysis, the peak positions may shift. The measurement error of 2θ values is sometimes about ±0.2°, and slight differences in 2θ measurements of XRPD patterns may occur between different instruments and samples. Therefore, the 2θ values should not be regarded as absolute. Accordingly, it is well known to those skilled in the art that this error should be taken into account when determining each crystalline structure. Therefore, in some embodiments, the crystals of the present invention are characterized by XRPD patterns having certain peak positions, substantially as shown in the XRPD patterns provided in the accompanying drawings of the present invention.

DSC measures the transition temperatures at which a crystal absorbs or releases heat due to changes in its crystalline structure or melting of the crystal. For the same crystal of the same compound, the error in the thermal transition temperatures and melting points in continuous analysis is typically within about 5° C. Accordingly, when a compound is described as having a given DSC peak or melting point, it means that the DSC peak or melting point is ±5° C. DSC provides an auxiliary method for distinguishing different crystals. Different crystalline forms can be identified based on their distinct transition temperature characteristics. It should be pointed out that, for mixtures, their DSC peaks or melting points may vary over a wider range. In addition, since decomposition may occur during the melting of materials, the melting temperature is related to the heating rate.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, halting progression of the disease or condition, relieving a condition caused by the disease or condition, or stopping the symptoms of the disease or condition either prophylactically and/or therapeutically.

The term “prevent” means administering a compound or formulation of the present invention to prevent one or more symptoms associated with the disease, and includes: preventing the occurrence of the disease or disease state in mammals, particularly in mammals susceptible to the disease state but not yet diagnosed as having the disease state.

“One embodiment” or “an embodiment” or “in another embodiment” or “in certain embodiments” mentioned throughout the specification means that specific reference elements, structures, or features related to that embodiment are included in at least one embodiment. Therefore, the phrases “in one embodiment” or “in an embodiment” or “in another embodiment” or “in certain embodiments” that appear at different positions throughout the specification do not necessarily refer to the same embodiment. Furthermore, the specific elements, structures, or features may be combined in any appropriate manner in one or more embodiments.

The term “acceptable” with respect to a formulation, composition or ingredient, as used herein, means having no persistent detrimental effect on the general health of the mammal being treated.

The terms “administer,” “administering”, “administration,” and the like, as used herein, refer to the methods that is used to enable delivery of compounds or compositions to the desired site of biological action. These methods include, but are not limited to oral routes, parenteral injection (including intravenous, subcutaneous, or infusion). Those of skill in the art are familiar with administration techniques that are employed with the compounds and methods described herein. In some embodiments, the compounds and compositions described herein are administered orally.

The term “subject” or “patient” encompasses mammals. Examples of mammals include, but are not limited to, any member of the Mammalian class: humans, non-human primates such as chimpanzees, and other apes and monkey species. In one aspect, the mammal is a human.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

In some embodiments, “therapeutically effective amount” refers to an amount of the compound of formula (I) that is administered to a human that is sufficient or effective to treat (e.g., halt, slow down the progression of, decrease the severity of) a disease associated with increased NSP enzyme activity, including alleviating symptoms of, or reducing the severity of symptoms of, such diseases. In some embodiments, “therapeutically effective amount” refers to an amount of the compound of formula (I) that is administered to a human with an airway inflammatory disease associated with increased NSP enzyme activity, particularly neutrophil elastase, that is sufficient or effective to treat (e.g., halt, slow down the progression of, decrease the severity of) the airway inflammatory disease including alleviating symptoms of, or reducing the severity of symptoms of, the airway disease. In some embodiments, “therapeutically effective amount” refers to an amount of the compound of formula (I) that is administered to a human with COPD or bronchiectasis that is sufficient or effective to treat (e.g., halt, slow down the progression of, decrease the severity of) COPD or bronchiectasis in the human, including alleviating symptoms of, or reducing the severity of symptoms of, COPD or bronchiectasis.

The terms “about” or “approximately” mean within an acceptable error range for the particular value as determined by one of ordinary skill in the art, which will depend in part on how the value is measured or determined, e.g., the limitations of the measurement system. Where particular values are described in the application and claims, unless otherwise stated the term “about” should be assumed to mean an acceptable error range for the particular value. In some embodiments, “about” means plus or minus 5% of the recited value, plus or minus 10% of the recited value, or plus or minus 15% of the recited value. In some embodiments, “about” means plus or minus 10% of the recited value.

### Active Ingredient

The active ingredient of the present invention is the compound of formula (I), particularly the compound of formula (I) in the form of crystalline forms of the present invention,

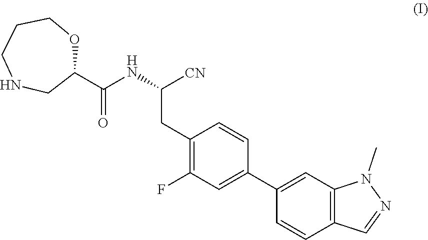 [SMILES: Cn1ncc2ccc(-c3ccc(C[C@@H](C#N)NC(=O)[C@@H]4CNCCCO4)c(F)c3)cc21]

for example crystalline form A, crystalline form B, crystalline form C, crystalline form D, or crystalline form E, or a combination thereof.

The compound of formula (I) is also known as (S)—N—((S)-1-cyano-2-(2-fluoro-4-(1-methyl-1H-indazol-6-yl)phenyl)ethyl)-1,4-oxazepane-2-carboxamide.

The compound of formula (I) of the present invention is a DPP1 inhibitor, thus the crystalline forms of the present invention can be used in the preparation of a DPP1 inhibitor or a medicament for preventing or treating a DPP1-related disease.

Preferably, in the active ingredient, the crystalline form A is present in an amount of >50 wt %, for example 50-100 wt %, preferably 80-99.99 wt % and more preferably 90-99.99 wt % or 98-99.99 wt %.

### Pharmaceutical Composition

The present invention further provides a pharmaceutical composition comprising any one or more of the crystalline forms according to the present invention, and a pharmaceutically acceptable carrier.

As used herein, “safe and effective amount” refers to the amount of a compound (or crystalline form) that is sufficient to significantly improve the condition without causing serious side effects. Generally, the pharmaceutical composition contains 1-2000 mg of a crystalline form of the present invention per dose, and more preferably 10-200 mg of a crystalline form of the present invention per dose. Preferably, the “dose” is a capsule or tablet.

“Pharmaceutically acceptable carrier” refers to one or more compatible solid or liquid fillers or gel materials which are suitable for use in humans and shall have sufficient purity and low toxicity. “Compatibility” herein refers to the ability of the components in the composition to be blended with the active ingredient of the present invention and with each other without significantly reducing the pharmaceutical efficacy of the active ingredient. Some examples of pharmaceutically acceptable carriers are cellulose and its derivatives (such as sodium carboxymethyl cellulose, sodium ethyl cellulose, and cellulose acetate), gelatin, talc, solid lubricants (such as stearic acid, and magnesium stearate), calcium sulfate, vegetable oils (such as soybean oil, sesame oil, peanut oil, and olive oil), polyols (such as propylene glycol, glycerol, mannitol, and sorbitol), emulsifying agents (such as Tween®), wetting agents (such as sodium dodecyl sulfate), coloring agents, flavoring agents, stabilizers, antioxidants, preservatives, and pyrogen-free water, etc.

The crystalline forms or pharmaceutical compositions of the present invention are not particularly limited in their routes of administration. Representative routes of administration include (but not limited to): oral and parenteral (intravenous, intramuscular or subcutaneous), and topical administration.

In some embodiments, the pharmaceutical composition is formulated for administration to a mammal by oral administration. In some embodiments, the pharmaceutical composition is in the form of a solid form pharmaceutical composition. In some embodiments, the pharmaceutical composition is in the form of a tablet, a pill, or a capsule.

Solid dosage forms for oral administration include capsules, tablets, pills, powders, and granules. In these solid dosage forms, the active ingredient is mixed with at least one conventional inert excipient (or carrier), such as sodium citrate or dicalcium phosphate, or with the following components: (a) fillers or bulking agents, such as microcrystalline cellulose, starch, lactose, sucrose, glucose, mannitol, and silicic acid; (b) binders, such as hydroxymethyl cellulose, alginate, gelatin, polyvinylpyrrolidone, sucrose, and Arabic gum; (c) humectants, such as glycerol; (d) disintegrating agents, such as agar, calcium carbonate, potato starch or cassava starch, alginic acid, certain complex silicates, sodium carbonate, cross-linked polyvinylpyrrolidone, cross-linked carboxymethyl cellulose sodium; (e) slow dissolving agents, such as paraffin wax; (f) absorption accelerators, such as quaternary ammonium compounds; (g) wetting agents, such as cetyl alcohol and glycerol monostearate; (h) adsorbents, such as kaolin; and (i) lubricants, such as talc, calcium stearate, magnesium stearate, solid polyethylene glycol, sodium dodecyl sulfate, or mixtures thereof. In capsules, tablets, and pills, dosage forms may also comprise buffering agents.

Solid dosage forms such as tablets, sugar pills, capsules, pills, and granules can be prepared using coatings and shell materials, such as enteric coatings and other materials well known in the art. They may comprise opacifying agents, and the release of the active ingredient in the composition can be delayed and occur in a particular part of the gastrointestinal tract. Examples of embedding components which can be used are polymeric substances and waxes. If necessary, the active ingredient can also be formulated in the form of microcapsules with one or more of the above-mentioned excipients.

Liquid dosage forms for oral administration include pharmaceutically acceptable emulsions, solutions, suspensions, syrups or tinctures. In addition to the active ingredient, the liquid dosage forms may comprise inert diluents commonly used in the art, such as water or other solvents, solubilizing agents, and emulsifying agents, such as ethanol, isopropanol, ethyl carbonate, ethyl acetate, propylene glycol, 1,3-butanediol, dimethylformamide, as well as oils, particularly cottonseed oil, peanut oil, corn germ oil, olive oil, castor oil, and sesame oil, or mixtures of these substances, etc.

In addition to these inert diluents, the composition may also comprise adjuvants such as wetting agents, emulsifying agents and suspending agents, sweetening agents, flavoring agents, and fragrances.

In addition to the active ingredient, the suspensions may comprise suspending agents such as ethoxylated isooctadecanol, polyoxyethylene sorbitol and sorbitan esters, microcrystalline cellulose, aluminium methoxide and agar, or mixtures of these substances.

Compositions for parenteral injection may comprise physiologically acceptable sterile aqueous or non-aqueous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

The dosage forms of the crystalline forms of the present invention for topical administration include ointments, powders, patches, sprays, and inhalants. The active ingredient is mixed under sterile conditions with physiologically acceptable carriers and any preservatives, buffering agents, or if necessary, with propellants that may be required.

The crystalline forms of the present invention may be administered alone or in combination with other pharmaceutically acceptable compounds.

When the pharmaceutical composition is used, a safe and effective amount of an crystalline form of the present invention is administered to a mammal in need of treatment (such as a human). The dose administered is a pharmaceutically effective dose. For a person weighing 60 kg, the daily dose is usually 1~2000 mg, preferably 10~500 mg. Of course, the specific dose should also take into account factors such as the route of administration and the patient's health condition, which are within the skill range of the skilled practitioners.

### Treatment Methods

In some embodiments, described herein is a method of treating an airway disease in a mammal comprising administering the compound of formula (I) to the mammal. In some embodiments, described herein is a method of treating an airway disease in a mammal comprising administering a therapeutically amount of a compound of formula (I) to the mammal. In some embodiments, the airway disease is COPD. In some embodiments, the airway disease is bronchiectasis. In some embodiments, the airway disease is non-cystic fibrosis bronchiectasis (NCFBE).

In some embodiments, the compound of formula (I) is used to treat an airway inflammatory disease in a human. In some embodiments, the compound of formula (I) is used to treat a neutrophil-driven airway disease in a human. In some embodiments, the compound of formula (I) is used to treat COPD in a human. In some embodiments, the compound of formula (I) is used to treat bronchiectasis in a human. In some embodiments, the compound of formula (I) is used to treat neutrophilic COPD in a human. In some embodiments, the compound of formula (I) is used to treat NCFBE in a human.

In some embodiments, described herein is a method of treating an airway inflammatory disease in a mammal comprising administering a pharmaceutical composition comprising the compound of formula (I) to the mammal. In some embodiments, described herein is a method of treating an airway inflammatory disease in a mammal comprising administering a therapeutically amount of a pharmaceutical composition comprising the compound of formula (I) to the mammal. In some embodiments, the airway inflammatory disease is COPD. In some embodiments, the airway inflammatory disease is bronchiectasis. In some embodiments, the airway inflammatory disease is non-cystic fibrosis bronchiectasis (NCFBE). In some embodiments, the compound of formula (I) is orally administered in the form of a solid form pharmaceutical composition comprising a crystalline form of the compound of formula (I), wherein the crystalline form of the compound of formula (I) is crystalline form A.

In some embodiments, described herein is a method of treating COPD in a human comprising orally administering a pharmaceutical composition comprising the compound of formula (I) to the human in need thereof. In some embodiments, described herein is a method of treating COPD in a human comprising orally administering a therapeutically amount of a pharmaceutical composition comprising a crystalline form of the compound of formula (I) to the human in need thereof. In some embodiments, described herein is a method of treating COPD in a human comprising orally administering a therapeutically amount of a solid form pharmaceutical composition comprising a crystalline form of the compound of formula (I) to the human in need thereof.

In some embodiments, described herein is a method of treating bronchiectasis in a human comprising orally administering a pharmaceutical composition comprising the compound of formula (I) to the human in need thereof. In some embodiments, described herein is a method of treating bronchiectasis in a human comprising orally administering a therapeutically amount of a pharmaceutical composition comprising a crystalline form of the compound of formula (I) to the human in need thereof. In some embodiments, described herein is a method of treating bronchiectasis in a human comprising orally administering a therapeutically amount of a solid form pharmaceutical composition comprising a crystalline form of the compound of formula (I) to the human in need thereof.

In some embodiments, described herein is a method of treating non-cystic fibrosis bronchiectasis (NCFBE) in a human comprising orally administering a pharmaceutical composition comprising the compound of formula (I) to the human in need thereof. In some embodiments, described herein is a method of treating NCFBE in a human comprising orally administering a therapeutically amount of a pharmaceutical composition comprising a crystalline form of the compound of formula (I) to the human in need thereof. In some embodiments, described herein is a method of treating NCFBE in a human comprising orally administering a therapeutically amount of a solid form pharmaceutical composition comprising a crystalline form of the compound of formula (I) to the human in need thereof.

In some embodiments, the pharmaceutical composition is a solid form pharmaceutical composition. In some embodiments, the solid form pharmaceutical composition is in the form of a tablet, a pill, or a capsule. In some embodiments, the solid form pharmaceutical composition is in the form of a tablet. In some embodiments, the solid form pharmaceutical composition is in the form of a pill. In some embodiments, the solid form pharmaceutical composition is in the form of a capsule. In some embodiments, the solid form pharmaceutical composition comprises crystalline form A of the compound of formula (I). In some embodiments, the solid form pharmaceutical composition comprises crystalline form A of the compound of formula (I). In some embodiments, the solid form pharmaceutical composition comprises a crystalline form of the compound of formula (I), wherein the crystalline form of the compound of formula (I) is crystalline form A, wherein the crystalline form A is substantially free of other crystalline forms of the compound of formula (I). In some embodiments, the solid form pharmaceutical composition comprises a crystalline form of the compound of formula (I), wherein the crystalline form of the compound of formula (I) is crystalline form A, wherein the crystalline form A is substantially free of crystalline form B, crystalline form C, crystalline form D, and crystalline form E of the compound of formula (I).

In some embodiments, treating COPD in a human with the compound of formula (I) comprises lowering the annualized rate of pulmonary exacerbations, slowing the decline of lung function, prolonging the time to the first exacerbation, lowering the frequency of severe exacerbations, or combinations thereof, as compared to a human with COPD not being treated with the compound of formula (I).

In some embodiments, treating COPD comprises reducing NSP activity levels in the plasma of the human with COPD.

In some embodiments, treating NCFBE in a human with the compound of formula (I) comprises lowering the annualized rate of pulmonary exacerbations, slowing the decline of lung function, prolonging the time to the first exacerbation, lowering the frequency of severe exacerbations, or combinations thereof, compared to a human with NCFBE not being treated with the compound of formula (I).

In some embodiments, treating NCFBE comprises reducing NSP activity levels in the plasma of the human with NCFBE.

In any of the aforementioned embodiments are further embodiments comprising single administrations of the effective amount of the compound of formula (I), including further embodiments in which (i) the compound of formula (I) is administered once a day; or (ii) the compound of formula (I) is administered to the mammal multiple times over the span of one day.

In any of the aforementioned embodiments are further embodiments comprising multiple administrations of the effective amount of the compound of formula (I), including further embodiments in which (i) the compound of formula (I) is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended, or the dose of the compound being administered is temporarily reduced; at the end of the drug holiday, dosing of the compound is resumed. In one embodiment, the length of the drug holiday varies from 2 days to 1 year.

In certain instances, it is appropriate to administer the compound of formula (I) in combination with one or more other therapeutic agents.

### The Main Advantages of the Present Invention Include

1. The present invention provides novel crystalline forms of the compound of formula (I) and preparation methods and uses thereof.
2. The crystalline form A of the present invention is easy to prepare, and compared to other crystalline forms, exhibits better crystallinity and a single melting point peak. It is an anhydrous crystalline form and has a short crystal transformation cycle, a high yield, as well as good physical and chemical stability, high purity, and low hygroscopicity, thereby demonstrating significant industrial application value and economic value, and promising prospects for pharmaceutical development.

The present invention is further illustrated in combination with the following specific Examples. It should be understood that these Examples are intended only to illustrate the present invention and not to limit the scope of the present invention. Experimental methods without specific conditions specified in the following Examples are generally carried out under the conventional conditions, or in accordance with the conditions recommended by the manufacturers. Percentages and parts are calculated by weight unless otherwise stated.

All solvents used in the present invention are commercially available and can be used without further purification.

The solvents used in the present invention are commercially available.

The following abbreviations are employed in the present invention: DCM represents dichloromethane; DMF represents N, N-dimethylformamide; DMSO represents dimethyl sulfoxide; EtOH represents ethanol; MeOH represents methanol; H₂O represents water; NMM represents N-methylmorpholine; TFAA represents trifluoroacetic anhydride; MTBE represents methyl tert-butyl ether; Exo Up indicates that exothermic peaks are upward in the DSC pattern.

Compounds are named according to conventional naming principles in the art or using ChemDraw® software, and commercially available compounds are given the names provided in the supplier catalogs.

### Instruments and Analytical Methods Thereof

Although the following diffractometer was used, other types of diffractometers could be used. Furthermore, other wavelengths could be used and converted to the Cu Kα. In some embodiments, Synchrotron Radiation X-Ray Powder Diffraction (SR-XRPD) can be used to characterize the crystalline forms.

X-ray powder diffractometer (XRPD) method 1 of the present invention: the instrument parameters are shown in Table 6.

TABLE 6
XRPD test parameters
XRPD test parameters

| Model | BRUKER D2 PHASER X-ray diffractometer |
|---|---|
| X-ray | Cu, Kα, Kα1 (Å): 1.54060, Kα2 (Å): 1.54439 |
| X-ray tube setting | 30 kV, 10 mA |
| Scan mode | continuous |
| Scan range (2θ(°)) | 3-60 |
| Scan time per step (s) | 1.10 |
| Scan step size (2θ(°)) | 0.020 |

X-ray powder diffractometer (XRPD) method 2 of the present invention: the X-ray diffraction pattern was acquired by the Bruker D2 Phaser instrument, and the instrument parameters are shown in Table 7.

TABLE 7
XRPD test parameters

| | 3 − 40 + 0.02 + | | Step |
|---|---|---|---|
| Method name | 0.2 + 15 · bsml | Induction mode | measurement |
| X-ray emitter | Cu, k-Alphal | Tube voltage | 30 |
| | (λ = 1.54184 Å) | (kV) | |
| Tube current (mA) | 10 | Divergence slit | 0.6 |
| | | (mm) | |
| Primary optical path | 2.5 | Secondary optical | 2.5 |
| axial Soller slit (°) | | path axial Soller | |
| | | slit (°) | |
| Detector slit (°) | 0.075 | Anti-scattering | 1 |
| | | slit (mm) | |
| Scan axis | θs-θd | Step size (deg) | 0.02 |
| Dwell time per step | 0.2 | Scan range (deg) | 3-40 |
| (S) | | | |

Thermal Gravimetric Analyzer (TGA) and Differential Scanning Calorimeter (DSC) method 1 for the crystalline forms of the present invention: the test parameters are shown in Table 8.

TABLE 8
TGA and DSC test parameters

| Parameters | TGA test parameters | DSC test parameters |
|---|---|---|
| Instrument model | Discovery TGA 5500 | Discovery DSC2500 |
| | Thermal Gravimetric | Differential Scanning |
| | Analyzer | Calorimeter |
| Method | Linear heating | Linear heating |
| Sample pan | Aluminum pan, open | Aluminum pan, |
| | | sealed/unsealed |
| Temperature range | 30-300° C. | 30-300° C. |
| Scan rate | 10 | 10 |
| (° C./minute) | | |
| Purge gas and | Nitrogen (25 mL/min) | Nitrogen (50 mL/min) |
| flow rate | | |

Thermal Gravimetric Analyzer (TGA) and Differential Scanning Calorimeter (DSC) method 2 for the crystalline forms of the present invention: the test parameters are shown in Table 9.

TABLE 9
TGA and DSC test parameters

| Parameters | TGA test parameters | DSC test parameters |
|---|---|---|
| Instrument model | TA 550 | TA 250 |
| Method | Linear heating | Linear heating |
| Sample pan | Aluminum pan, open | Aluminum pan, |
| | | sealed/unsealed |
| Temperature range | 25° C. - temperature | 25° C. - temperature |
| | of the set endpoint | of the set endpoint |
| Scan rate | 10 | 10 |
| (° C./minute) | | |
| Purge gas | Nitrogen | Nitrogen |

Dynamic Vapor Sorption (DVS) method for the crystalline forms of the present invention was carried out using the DVS Intrinsic plus instrument (Surface Measurement Systems, UK). The relative humidity at 25° C. was corrected using the deliquescence points of lithium chloride (LiCl), magnesium nitrate [Mg(NO₃)₂] and potassium chloride (KCl). The test parameters are shown in Table 10.

TABLE 10
DVS test parameters

| Parameters | Set value | |
|---|---|---|
| Temperature | 25° | C. |
| Purge gas and flow rate | N₂, 200 mL/min | |
| Solvent | Water | |
| Sample mass | 30-50 | mg |
| Equilibrium | 25° C., dm/dt <0.002% | |
| Minimum equilibrium time | 30 | min |
| Maximum equilibrium time | 360 | min |
| RH range | 0% RH-90% RH-0% RH | |
| RH gradient | 10% | RH |

### Example 1: Preparation of the Compound of Formula (I) and the Crystalline Form a

### Synthetic Route:

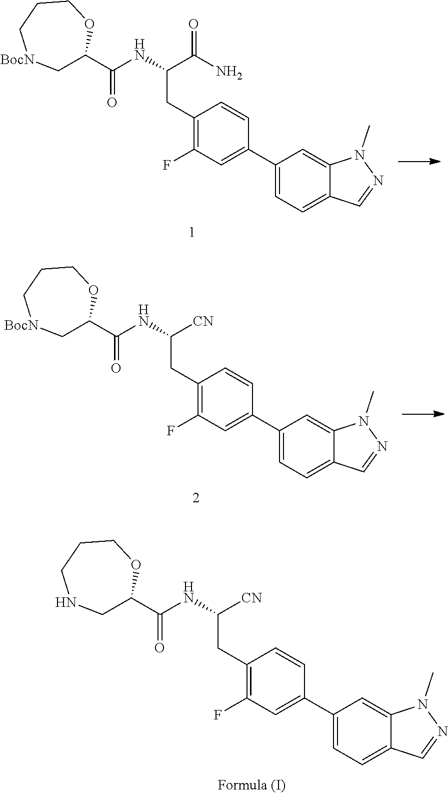 [SMILES: Cn1ncc2ccc(-c3ccc(C[C@H](NC(=O)[C@@H]4CN(C(=O)OC(C)(C)C)CCCO4)C(N)=O)c(F)c3)cc21 ; Cn1ncc2ccc(-c3ccc(C[C@@H](C#N)NC(=O)[C@@H]4CN(C(=O)OC(C)(C)C)CCCO4)c(F)c3)cc21 ; Cn1ncc2ccc(-c3ccc(C[C@@H](C#N)NC(=O)[C@@H]4CNCCCO4)c(F)c3)cc21]

### Step 1

Compound 1 (149 g, 276.13 mmol) was dissolved in DCM (1500 mL), and NMM (111.72 g, 1.10 mol) was added at 0° C., followed by dropwise addition of TFAA (115.99 g, 552.26 mmol). After the addition was complete, the reaction was warmed to 25° C. and stirred for 12 hours. Water (1000 mL) was added to the reaction mixture with stirring, and the mixture was allowed to stand to separate dichloromethane. The organic phase was washed with saturated aqueous sodium chloride solution (1000 mL*2), dried over anhydrous sodium sulfate, filtered, and concentrated under reduced pressure and dried to afford compound 2.

### Step 2

Compound 2 (139 g, 266.50 mmol) was dissolved in formic acid (1000 mL) and H₂O (100 mL) was added with stirring. The reaction was stirred at 25° C. for 12 hours. The reaction mixture was directly concentrated under reduced pressure, and the residue was dissolved in water (1 L). Dichloromethane (2 L) was added, and the mixture was stirred for 5 minutes. The pH was adjusted to 8 with 25% ammonia water and the mixture was stirred for 10 minutes until a clear solution was obtained, and the dichloromethane phase was separated. The organic phase was concentrated under reduced pressure until a small amount of solvent remained, and isopropanol (1000 mL) was added, followed by concentration under reduced pressure and drying. The resulting solid was stirred in isopropanol (1500 mL) at 50° C. for 2 hours, slowly cooled to 25° C. and stirred for 1 hour, and filtered to afford the compound of formula (I).

The compound of formula (I) obtained as described above was stirred in a mixed solvent of isopropanol (1500 mL) and water (100 mL) at 50° C. for 2 hours, and slowly cooled to 25° C. and stirred for 1 hour, and filtered, and the filter cake was dried under vacuum (40° C.) to obtain a white solid product, which is the crystalline form A of the compound of formula (I). The e.e. value was determined via SFC (column: Chiralcel AD-3 50 mm×4.6 mm×3 m; mobile phase: phase A was supercritical CO₂, phase B was ethanol solution containing 0.05% diethylamine; gradient: 5%-40% phase B) for the compound of formula (I): e.e. %=100%, RT=2.091 min. HPLC (chromatographic column: Ultimate LP-C18, 150×4.6 mm, 5 μm; mobile phase: phase A was aqueous solution containing 0.04% trifluoroacetic acid and phase B was acetonitrile solution containing 0.02% trifluoroacetic acid; gradient: 10%-80% phase B, elution time of 10 minutes, holding at 80% for 5 minutes; flow rate: 1.5 mL/min; column temperature: 50° C.; wavelength: UV 220 nm) test showed a purity of 98.8%, RT=7.913. MS-ESI calculated for [M+H]⁺: 422, found 422.

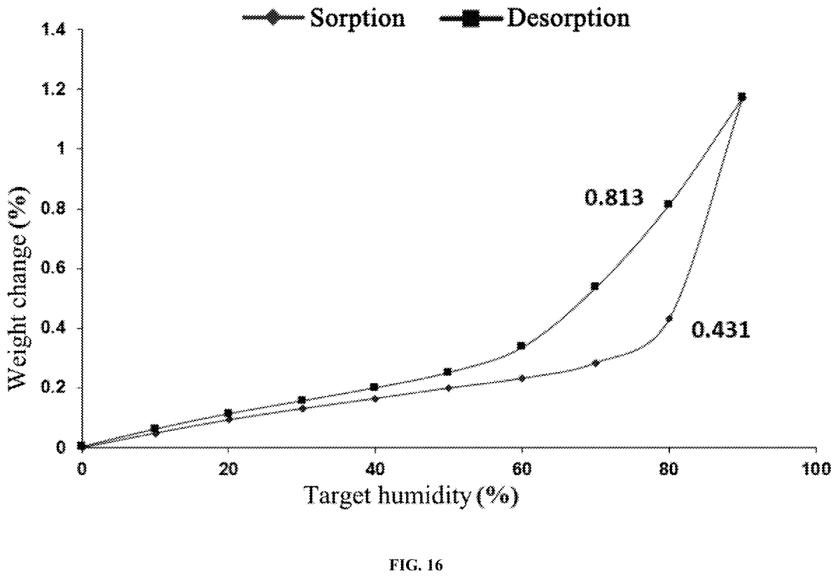

The XRPD of the crystalline form A of the compound of formula (I) was tested using the corresponding method 1, and DSC and TGA were tested using the corresponding method 1. The results of XRPD, DSC, and TGA detection are shown in FIGS. 1, 2, and 3, respectively, and the DVS detection results are shown in FIG. 16. The DSC characterization results showed one endothermic peak, and the TGA results showed no weight loss before reaching 100° C., indicating that the crystalline form A of the compound of formula (I) is an anhydrous crystalline form.

### Example 2: Preparation of the Crystalline Form B of the Compound of Formula (I)

The compound of formula (I) (40.71 mg) was weighed into a glass bottle and 1 mL of ethyl acetate was added. After stirring at 50° C. for 3 days, the sample was filtered. The resulting solid was purged with nitrogen for 4 h to afford the crystalline form B of the compound of formula (I). The XRPD of the crystalline form B of the compound of formula (I) was tested using the corresponding method 2, and DSC and TGA were tested using the corresponding method 2. The results of XRPD, DSC, and TGA detection are shown in FIGS. 4, 5, and 6, respectively.

### Example 3: Preparation of the Crystalline Form C of the Compound of Formula (I)

The compound of formula (I) (90.58 mg) was weighed into a glass bottle and 2 mL of ethanol was added. The mixture was stirred at 50° C. to obtain a clear solution and then filtered. The filtrate was cooled from 50° C. to 25° C. (at a rate of 0.2° C./min) and stirred for 16 hours, during which no solid precipitated. Then the filtrate was cooled to −10° C. and stirred for 20 h, after which a solid precipitated and was filtered. The resulting solid was purged with nitrogen for 4 h to afford the crystalline form C of the compound of formula (I). The XRPD of the crystalline form C of the compound of formula (I) was tested using the corresponding method 2, and DSC and TGA were tested using the corresponding method 2. The results of XRPD, DSC, and TGA detection are shown in FIGS. 7, 8, and 9, respectively. The TGA results showed a weight loss of 6.495% at 25-100° C. ¹H NMR results showed a residue of 6.9% ethanol (0.7 mol). The crystalline form C of the compound of formula (I) remained unchanged after heating to 85° C. and 120° C. Therefore, it can be inferred that the crystalline form C of the compound of formula (I) is a solvate (0.7 mol ethanol).

### Example 4: Preparation of the Crystalline Form D of the Compound of Formula (I)

The compound of formula (I) (30.92 mg) was weighed into a glass bottle and 1 mL of methanol was added. After the solution became clear, it was filtered and then allowed to evaporate slowly at room temperature (15-22° C.) to afford the crystalline form D of the compound of formula (I). The XRPD of the crystalline form D of the compound of formula (I) was tested using the corresponding method 2, and DSC and TGA were tested using the corresponding method 2. The results of XRPD, DSC, and TGA detection are shown in FIGS. 10, 11, and 12, respectively. The results of DSC showed two endothermic peaks and TGA results showed a weight loss of 2.273% at 23-120° C. ¹H NMR results showed no residual solvent. After heating to 115° C., the crystalline form D transformed into the crystalline form A. Therefore, it is inferred that the crystalline form D of the compound of formula (I) is a hydrate (0.5 mol).

### Example 5: Preparation of the Crystalline Form E of the Compound of Formula (I)

The compound of formula (I) (405.10 mg) was weighed into a glass bottle and 12.5 mL of ethanol was added. The mixture was stirred at 70-80° C. to obtain a clear solution and then filtered. The filtered solution was cooled from 25° C. to −10° C. (at a rate of 0.2° C./min), and then stirred for 24 h at −10° C. A sample was taken and analyzed by XRPD, and the results showed that it was the crystalline form C of the compound of formula (I). The mixture was further stirred at −10° C. for additional 24 h and then filtered. The filter cake was dried under vacuum at 50° C. for 20 h to afford the crystalline form E of the compound of formula (I). The XRPD of the crystalline form E of the compound of formula (I) was tested using the corresponding method 2, and DSC and TGA were tested using the corresponding method 2. The results of XRPD, DSC, and TGA detection are shown in FIGS. 13, 14, and 15, respectively. The crystalline form E of the compound of formula (I) remained unchanged after heating to 80° C. and transformed into the crystalline form A of the compound of formula (I) after heating to 120° C. The DSC characterization results showed one endothermic peak and one exothermic peak, and the TGA results showed a weight loss of 0.293% at 30-100° C. ¹H NMR results showed no residual solvent. Therefore, it can be inferred that the crystalline form E of the compound of formula (I) is an anhydrate.

### Example 6: Hygroscopicity Study of the Crystalline Form a of the Compound of Formula (I)

Experimental materials: DVS intrinsic plus dynamic vapor sorption instrument (SMS, UK).

Experimental method: 30-50 mg of the crystalline form A of the compound of formula (I) was taken, placed in the DVS sample pan and tested.

Reference for hygroscopicity assessment: Guiding principles for drug hygroscopicity testing in the Part IV of Pharmacopoeia of the People's Republic of China (2015 Edition). The classification of hygroscopicity assessment is shown in Table 11 below:

TABLE 11
Description of hygroscopicity characteristics
and definition of hygroscopic weight gain

| Hygroscopicity | |
|---|---|
| classification | ΔW % |
| Deliquescence | Absorption of sufficient |
| | moisture to form liquid |
| Extreme hygroscopicity | ΔW % ≥ 15% |
| hygroscopicity | 15% > ΔW % ≥ 2% |
| Slight hygroscopicity | 2% > ΔW % ≥ 0.2% |
| No or almost no | ΔW % < 0.2% |
| hygroscopicity | |

Note: ΔW % represents the hygroscopic weight gain of the tested sample at 25±1° C. and 80±2% RH.

Experimental results: The DVS pattern of the crystalline form A of the compound of formula (I) is shown in FIG. 16. DVS results showed that the sample exhibited a hygroscopic weight gain of 0.431% at a 25° C./80% RH condition, indicating slight hygroscopicity of the sample. XRPD test results showed that the crystalline form of the sample remained unchanged before and after the DVS test.

Experimental conclusion: The crystalline form A of the compound of formula (I) exhibited slight hygroscopicity at 25±1° C. and 80±2% RH and the crystalline form remained unchanged before and after the DVS test.

### Example 7: Competitive Slurry Experiment

Saturated solutions of the crystalline form A of the compound of formula (I) in ethanol, n-heptane, isopropanol, and MTBE were formulated at 25° C. and 50° C., respectively.

10 mg of the crystalline form A of the compound of formula (I) and 10 mg of the crystalline form E of the compound of formula (I) were weighted and added to the same saturated solution at the corresponding temperature. Samples were taken after stirring for 1 day and 4 days, respectively, and analyzed by XRPD. The experimental results are shown in Table 12.

TABLE 12
Competitive slurry experiment results

| | Crystalline | Crystalline | | | XRPD results-Day | XRPD results- |
|---|---|---|---|---|---|---|
| No. | form A/mg | form E/mg | Temperature/° C. | Solvent | 1 | Day 4 |
| 1 | 9.68 | 9.71 | 25 | ethanol | Crystalline form A | Crystalline form A |
| 2 | 9.93 | 10.27 | 25 | n-heptane | Crystalline form A | Crystalline form A |
| 3 | 9.80 | 9.88 | 25 | isopropanol | Crystalline form A | Crystalline form A |
| 4 | 9.83 | 10.20 | 25 | MTBE | Crystalline form A | Crystalline form A |
| 5 | 9.96 | 10.3 | 50 | ethanol | Crystalline form A | Crystalline form A |
| 6 | 9.66 | 10.20 | 50 | n-heptane | Crystalline form A | Crystalline form A |
| 7 | 10.24 | 10.03 | 50 | isopropanol | Crystalline form A | Crystalline form A |
| 8 | 9.71 | 10.23 | 50 | MTBE | Crystalline form A | Crystalline form A |

Experimental conclusion: The results of the competitive slurry experiment showed that the crystalline form A of the compound of formula (I) was obtained in all cases. The crystalline form A is more stable than the crystalline form E, further indicating that the crystalline form A is a stable crystalline form.

### Biological Activity Assessment

The biological activity of the compound of formula (I) was previously disclosed in PCT/CN2022/074088 (see biological activity of Compound 8, which was shown to exhibit potent inhibitory activity against DPP1 enzyme and exhibit good inhibitory activity against DPP1 in U937 cells, good pharmacokinetic properties in CD-1 mice and SD mice, high distribution in the bone marrow of CD-1 mice and SD mice, and can significantly inhibit the activity of neutrophil elastase in rats in vivo).

All references mentioned in the present invention are incorporated herein by reference, as if each reference was individually incorporated by reference. In addition, it should be understood that, after reading the above disclosure of the present invention, those skilled in the art may make various changes or modifications to the present invention, and these equivalent forms shall fall within the defined scope of the claims appended to this application.

What is claimed is:

1. A crystalline form of the compound of formula (I), wherein the crystalline form is crystalline form A, crystalline form B, crystalline form C, crystalline form D, or crystalline form E; 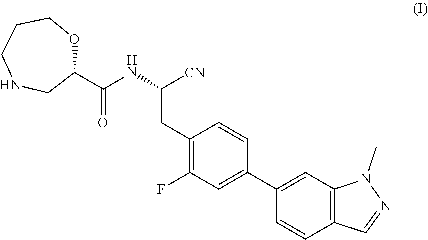 [SMILES: Cn1ncc2ccc(-c3ccc(C[C@@H](C#N)NC(=O)[C@@H]4CNCCCO4)c(F)c3)cc21]
wherein an X-ray powder diffraction pattern of the crystalline form A has diffraction peaks at the following 2θ angles: 13.822±0.200°, 15.236±0.200°, 16.899±0.200°, and 20.673±0.200°, as measured using Cu (Kα) radiation;
an X-ray powder diffraction pattern of the crystalline form B has diffraction peaks at the following 2θ angles: 8.954±0.200°, 13.530±0.200°, 15.480±0.200°, and 18.122±0.200°, as measured using Cu (Kα) radiation;
an X-ray powder diffraction pattern of the crystalline form C has diffraction peaks at the following 2θ angles: 8.069±0.200°, 11.606±0.200°, 13.120±0.200°, and 19.812±0.200°, as measured using Cu (Kα) radiation;
an X-ray powder diffraction pattern of the crystalline form D has diffraction peaks at the following 2θ angles: 9.105±0.200°, 15.206±0.200°, and 16.904±0.200°, as measured using Cu (Kα) radiation;
an X-ray powder diffraction pattern of the crystalline form E has diffraction peaks at the following 2θ angles: 9.331±0.200°, 12.051±0.200°, and 15.214±0.200°, as measured using Cu (Kα) radiation.

2. The crystalline form of claim 1, wherein the crystalline form is crystalline form A.

3. The crystalline form of claim 2, wherein the X-ray powder diffraction pattern of the crystalline form has diffraction peaks at the following 2θ angles: 11.716±0.200°, 13.822±0.200°, 15.236±0.200°, 16.899±0.200°, 20.673±0.200°, and 24.134±0.200°, as measured using Cu (Kα) radiation.

4. The crystalline form of claim 2, wherein the X-ray powder diffraction pattern of the crystalline form has diffraction peaks at the following 2θ angles: 10.267±0.200°, 11.716±0.200°, 13.822±0.200°, 15.236±0.200°, 16.899±0.200°, 17.550±0.200°, 18.110±0.200°, 20.673±0.200°, 24.134±0.200°, as measured using Cu (Kα) radiation.

5. The crystalline form of claim 2, wherein the X-ray powder diffraction pattern of the crystalline form has diffraction peaks at the following 2θ angles: 10.267±0.200°, 11.716±0.200°, 13.822±0.200°, 15.236±0.200°, 16.899±0.200°, 17.550±0.200°, 18.110±0.200°, 19.863±0.200°, 20.673±0.200°, 23.025±0.200°, 23.513±0.200°, and 24.134±0.200°, as measured using Cu (Kα) radiation.

6. The crystalline form of claim 1, wherein the crystalline form A has one or more characteristics selected from the group consisting of:
an XRPD pattern substantially as shown in FIG. 1;
a DSC pattern substantially as shown in FIG. 2; and/or
a TGA pattern substantially as shown in FIG. 3.

7. The crystalline form of claim 1, wherein the crystalline form A has one or more characteristics selected from the group consisting of:
a differential scanning calorimetry curve having a peak value of an endothermic peak at 141.01±3° C.; and/or
a thermogravimetric analysis curve showing a weight loss of 0.040% at 150.0±3° C.

8. A crystalline form of the compound of formula (I), wherein the crystalline form is crystalline form A; 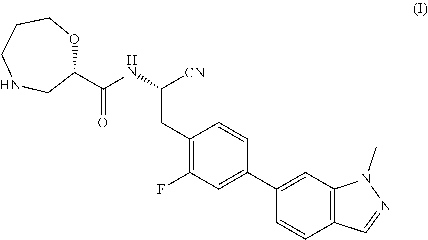 [SMILES: Cn1ncc2ccc(-c3ccc(C[C@@H](C#N)NC(=O)[C@@H]4CNCCCO4)c(F)c3)cc21]
wherein the crystalline form A is characterized as having X-ray powder diffraction pattern with peaks at the following 2θ angles: 13.822±0.200°, 15.236±0.200°, 16.899±0.200°, and 20.673±0.200°, as measured using Cu (Kα) radiation; and one or both of the following:
a differential scanning calorimetry curve having a peak value of an endothermic peak at 141.01±3° C.; or
a thermogravimetric analysis curve showing a weight loss of 0.040% at 150.0±3° C.

9. The crystalline form of claim 8, wherein the crystalline form A is characterized as having X-ray powder diffraction pattern with peaks at the following 2θ angles: 13.822±0.200°, 15.236±0.200°, 16.899±0.200°, and 20.673±0.200°, as measured using Cu (Kα) radiation; and a differential scanning calorimetry curve having a peak value of an endothermic peak at 141.01±3° C.

10. The crystalline form of claim 8, wherein the crystalline form A is characterized as having X-ray powder diffraction pattern with peaks at the following 2θ angles: 13.822±0.200°, 15.236±0.200°, 16.899±0.200°, and 20.673±0.200°, as measured using Cu (Kα) radiation; and a thermogravimetric analysis curve showing a weight loss of 0.040% at 150.0±3° C.

11. A crystalline form of the compound of formula (I), wherein the crystalline form is crystalline form A; 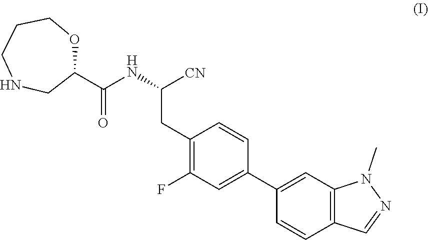 [SMILES: Cn1ncc2ccc(-c3ccc(C[C@@H](C#N)NC(=O)[C@@H]4CNCCCO4)c(F)c3)cc21]
wherein the crystalline form A is characterized as having an XRPD pattern substantially as shown in FIG. 1; and
a DSC pattern substantially as shown in FIG. 2; and/or
a TGA pattern substantially as shown in FIG. 3.

12. The crystalline form of claim 11, wherein the crystalline form A is characterized as having an XRPD pattern substantially as shown in FIG. 1; and
a DSC pattern substantially as shown in FIG. 2.

13. The crystalline form of claim 11, wherein the crystalline form A is characterized as having an XRPD pattern substantially as shown in FIG. 1; and
a TGA pattern substantially as shown in FIG. 3.

14. A pharmaceutical composition comprising the crystalline form of claim 1, and a pharmaceutically acceptable carrier.

15. The pharmaceutical composition of claim 14, wherein the pharmaceutical composition is a solid form pharmaceutical composition.

16. The pharmaceutical composition of claim 15, wherein the solid form pharmaceutical composition is a tablet, pill, or capsule.

17. A method for preparing the crystalline form of the compound of formula (I) of claim 1 comprising the steps of:
stirring the compound of formula (I) in a mixed solvent of isopropanol and water until a solid precipitates, filtering the solid, and drying the solid at 20-45° C. (preferably 30-40° C.) to obtain the crystalline form A.

18. A method of treating an airway inflammatory disease in a human comprising administering to the human in need thereof a therapeutically effective amount of the crystalline form of claim 1.

19. The method of claim 18, wherein the therapeutically effective amount of the crystalline form is administered in the form of a solid form pharmaceutical composition.

20. The method of claim 19, wherein the solid form pharmaceutical composition is a tablet, pill, or capsule.

21. The method of claim 18, wherein the airway inflammatory disease is chronic obstructive pulmonary disease (COPD), bronchiectasis, asthma, acute respiratory distress syndrome, exercise-induced bronchoconstriction (EIB), chronic bronchitis, emphysema, alpha-1 antitrypsin deficiency, combined pulmonary fibrosis and emphysema (CPFE), or asthma-COPD overlap syndrome (ACOS).

22. The method of claim 21, wherein the asthma is allergic asthma, aspirin-induced asthma, cough-variant asthma, exercise-induced asthma, nighttime asthma, steroid-resistant asthma, occupational asthma, eosinophilic asthma, neutrophilic asthma, or mixed eosinophilic and neutrophilic asthma.

23. The method of claim 18, wherein the airway inflammatory disease is chronic obstructive pulmonary disease (COPD).

24. The method of claim 18, wherein the airway inflammatory disease is bronchiectasis.

25. A method of treating chronic obstructive pulmonary disease (COPD) or bronchiectasis in a human comprising administering to the human in need thereof a therapeutically effective amount of the crystalline form of claim 8.

26. The method of claim 25, wherein the therapeutically effective amount of the crystalline form is administered in the form of a solid form pharmaceutical composition.

27. The method of claim 26, wherein the solid form pharmaceutical composition is a tablet, pill, or capsule.
